(19) United States
(12) Patent Application Publication
(10) Pub. No.: US 2005/0250100 A1
(43) Pub. Date: Nov. 10, 2005

(54) Method of utilizing the 5'end of transcribed nucleic acid regions for cloning and analysis

(76) Inventors: Yoshihide Hayashizaki, Tsukuba-shi, Ibaraki (JP); Piero Carninci, Wako-shi, Saitama (JP); Matthias T. Harbers, Tokyo (JP)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/517,544

(22) PCT Filed: Jun. 12, 2003
(86) PCT No.: PCT/JP03/07514

§ 371 Date: Jun. 9, 2005

(30) Foreign Application Priority Data
Jun. 12, 2002 (JP) 2002-171851
Aug. 12, 2002 (JP) 2002-235294

(51) Int. Cl. C12Q 1/68; C12P 19/34

(52) U.S. Cl. 435/6; 435/91.2

(57) ABSTRACT

A method is disclosed for obtaining the 5′ends of transcribed regions from a plurality of nucleic acid fragments obtained from biological materials or synthetic pools. DNA fragments encoding the 5′ends are enriched for their individual analysis or for the analysis of concatemers thereof. The sequence information derived from 5′ ends can be used for characterization and cloning of the transcriptome.

### TECHNICAL FIELD

The present invention relates to a method for selectively collecting multiple nucleic acid fragments containing information on the nucleotide sequences at the 5′ end of multiple mRNAs in a sample.

### BACKGROUND ART

In order to utilize genomic information, parts of the genome are transcribed into mRNA. For the understanding of the genome and its use in regulatory processes, information on individual mRNA species is required. Such information should include partial or full-length nucleotide sequences and their relative or absolute quantities in a given biological context.

Conventionally, the base sequences of mRNAs contained in a cell, tissue or organism have been analyzed by preparing a cDNA library through reverse transcription. The mRNAs are used as templates and individual cDNA fragments in said cDNA library are investigated. Since a sample contains a large number of various mRNAs, the conventional method is of limited efficiency in analyzing gene expression profiles and identifying rare genes. Therefore, other technologies have been developed to monitor the expression patterns of mRNA in complex samples and identify genes by short sequence elements called tags.

High-throughput expression profiling is commonly performed using so-called DNA microarrays (Jordan B., DNA Microarrays: Gene Expression Applications, Springer-Verlag, Berlin Heidelberg New York, 2001; and Schena A, DNA Microarrays, A Practical Approach, Oxford University Press, Oxford 1999). For such experiments, specific probes representing individual genes or transcripts are placed on a support and simultaneously hybridized with a plurality of samples. Positive signals will be obtained if a probe on the support reacts with a molecule presented with the sample. These experiments allow the parallel analysis of a large number of genes or transcripts. However, the approach is limited in that only genes or transcripts which have initially been identified by other experimental means can be studies. Such means can include cDNA libraries, partial sequence tags and/or results obtained from computer predictions. Due to this limitation of DNA microarray experiments, alternative approaches based on partial sequences or tags obtained from a plurality of mRNA samples are in use for gene discovery and expression profiling.

The so-called SAGE (Serial Analysis of Gene Expression) method is known as an efficient method of obtaining partial information on the base sequences in mRNAs (Velculescu V. E. et at., Science 270, 484-487 (1995)). According to this method, DNA concatemers are formed by ligating multiple short DNA fragments (initially about 10 bp) containing information on the base sequences at the 3′ end of multiple mRNAs, and the base sequences in these DNA concatemers are determined. This is a method for obtaining partial information on the base sequences at the 3′ end of multiple mRNAs. When only a short base sequence close to the 3′ end is available but the mRNAs itself is already known, the SAGE method can often identify a specific mRNA or gene, although the available base sequence is often as short as about 10 bp. Recently, an improved version of SAGE, the so-called LongSAGE, has been published. This method allows for the cloning of longer SAGE tags (Saha S. et al., Nat. Biotechnol. 20, 508-12 (2002), U.S. patent publication Nos. 20030008290 and 20030049653). The SAGE method is currently in wide use as an important method for analyzing genes expressed in specific cells, tissues or organisms, and SAGE tags are available for reference in the public domain, e.g. under http://cgap.nci.nih.gov/SAGE.

While the SAGE method can be used to learn a partial base sequence at the 3′ end of mRNAs, it is difficult to clone new genes based on the information in such short sequences at the 3′ end only. Despite its multiple applications, SAGE does not teach how to obtain cDNA clones close to the 5′ end of mRNAs. In fact, 4 bp restriction enzymes of Class IIS are used. A 4 bp cutter usually cleaves on average a few hundred nucleotides, which is on average one tenth of the average size of an mRNA transcript. Thus SAGE principles strongly suggest that 3′ ends are collected with high prevalence, and no information can be collected about the 5′ end for most of the transcripts. In addition, the initial version of SAGE was limited due to the short length of the tags, in most cases only tags of 10 bp lengths were used, and a reliable analysis and annotation of the information were not possible.

Although techniques exsit for the collection of full-length cDNA clones and sequences derived thereof, those are focusing on collecting the full-length cDNA clones and not fragments covering the 5′ ends only. Full-length cDNA cloning approaches are therefore not suitable for high throughput identification and analysis of start sites of transcription and the related promoter regions.

### SUMMARY OF THE INVETION

Accordingly, it is an object of the present invention to provide a new general method that enables the acquisition of information on the base sequences at 5′ ends of mRNAs in a sample. It is another object of the present invention to make it possible to clone new genes and analize genomic sequence information which relates to coding and regulatory regions. The information may include statistics on the transcriptional start sites derived from large numbers of 5′ end sequences.

Thus, the present invention refers generally to the concept of isolating portions of nucleic acids corresponding to the 5′end of transcribed genes and using them to further high-throughput analysis such as sequencing. The present invention offers a novel way to combine contrasting teachings and provide a new, high throughput approach to 5′ ends which is useful for promoter mapping and analysis. The method of the present invention is effective for analyzing the mRNAs contained in the sample for discovering and cloning of new genes and studying gene regulation. The use of the present invention to study and analyze complex regulatory networks in combination with the ability to identify and clone new genes opens a wide area of applications for monitoring biological systems and their status in development, homeostasis, disease, and beyond.

The present invention provides a new method for promoter analysis using 5′ ends, while SAGE does not allow any promoter analysis due to the use of unrelated 3′ ends.

After devoted research, the present inventors have completed the present invention by arriving at the fact that by selectively collecting multiple nucleic acid fragments containing information on the base sequences at the 5′ end of the mRNAs, it is not only possible to acquire information on the base sequences in mRNAs, but it is also possible to clone new genes; and they also have found a concrete method for attaining this goal.

That is, the present invention provides a method for preparing concatemers of a plurality of nucleic acid fragments related to nucleotide sequences of 5′ end regions of a plurality of mRNAs in a sample, comprising: a first step of selectively collecting a plurality of first-strand cDNAs which contain sequences complementary to 5′ end regions of mRNAs from cDNAs that have been formed using mRNAs present in the sample as templates; a second step of obtaining fragments of the first-strand cDNAs collected in the first step; a third step of selectively collecting fragments which contain at least sequences complementary to the 5′ end regions of said mRNAs; and a fourth step of ligating the collected fragments individually or in the form of a concatemer.

The present invention further provides a method for preparing concatemers of a plurality of nucleic acid fragments related to nucleotide sequences of 5′ end regions of a plurality of mRNAs in a sample, comprising: a first step of obtaining fragments of full-length cDNAs; a second step of selectively collecting fragments which contain at least sequences complementary to the 5′ end regions of said mRNAs; and a third step of ligating the collected fragments to form a concatemer. The present invention still further allows for the fractionation or isolation of the 5′ end sequences before cloning and sequencing in such cases first-strand cDNAs can be separated by subtractive hybridizations using drivers holding pluralities of nucleic acids of biological or artificial content. The present invention may be used for the identification of differentially expressed genes.

The present invention also provides a method for determining nucleotide sequences of 5′ end regions of a plurality of mRNAs by sequencing concatemers prepared by the method according to the present invention. By using concatemers to obtain information on a large number of 5′end sequence tags as presented in the invention, it is possible to effectively map transcriptional start sites and the related promoter sequences.

The present invention still further provides concatemers prepared by the method according to the present invention. The present invention still further provides a vector comprising said concatemer according the present invention. The present invention still further provides sequence tags derived from said concatemers prepared according to the present invention. The present invention still further provides means to use the sequences derived from said concatemers to analyze the content of the plurality of a RNA sample. The present invention still further provides means to use the sequences derived from said concatemers to identify regions in the genome, which are required for gene regulation and gene expression.

The invention is not limited to the use of concatemers for sequencing of 5′ ends, and modifications at particular steps for the enrichment of 5′ ends and their cloning as disclosed here allow for the individual sequencing of specific 5′ ends. Such embodiments of the invention would include a modification of the first and second steps, in which a linker that is specifically bound to a solid matrix is used. The cDNA bound to the support would then be used to prepare the sequencing reactions.

### BRIEF DESCRIPTION OF THE DRAWINGS

FIG. 1 shows expamplary principle workflows according to the present invention, following procedures described in the examples.

FIG. 2 shows an example of principle workflow of the invention given for the cloning of 5′ end specific tags into concatemers.

FIG. 3 shows a principle workflow according to the present invention to illustrate an alternative approach for the direct sequencing of 5′ end tags.

FIG. 4 shows examples for the ligation of the first linker for the cloning of 5′ end specific tags are presented. The examples specify the linkers used according to the protocols described in Examples 1 to 3.

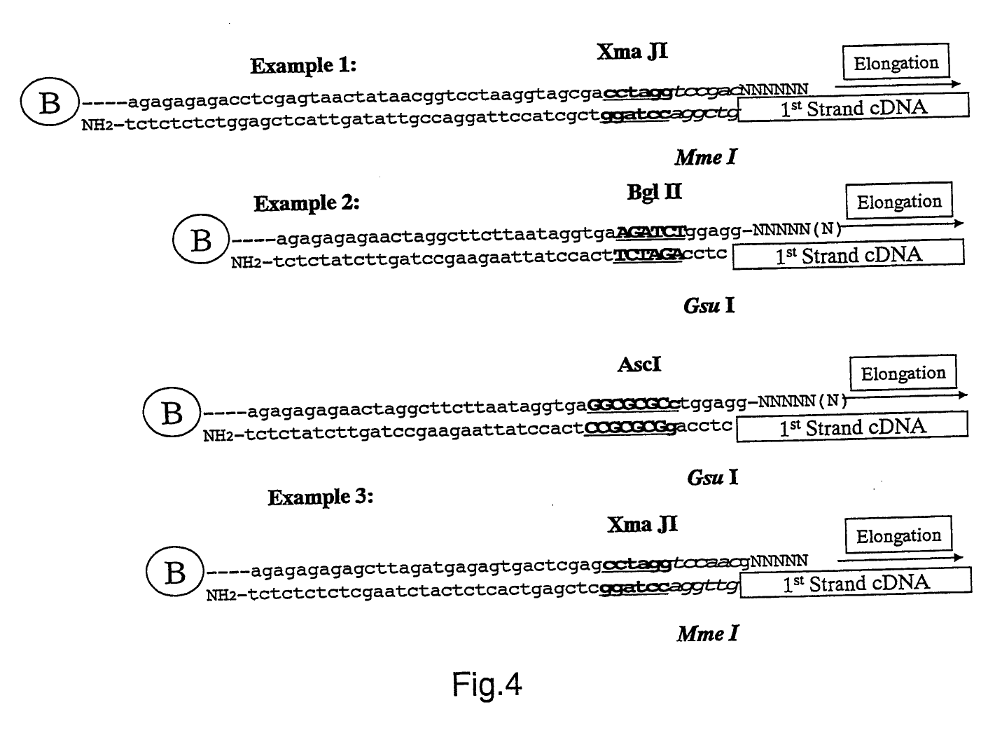

FIG. 5 shows examples for the ligation of the second linker for the cloning of 5′ end specific tags are presented. The examples specify the linkers used according to the protocols described in Examples 1 to 3.

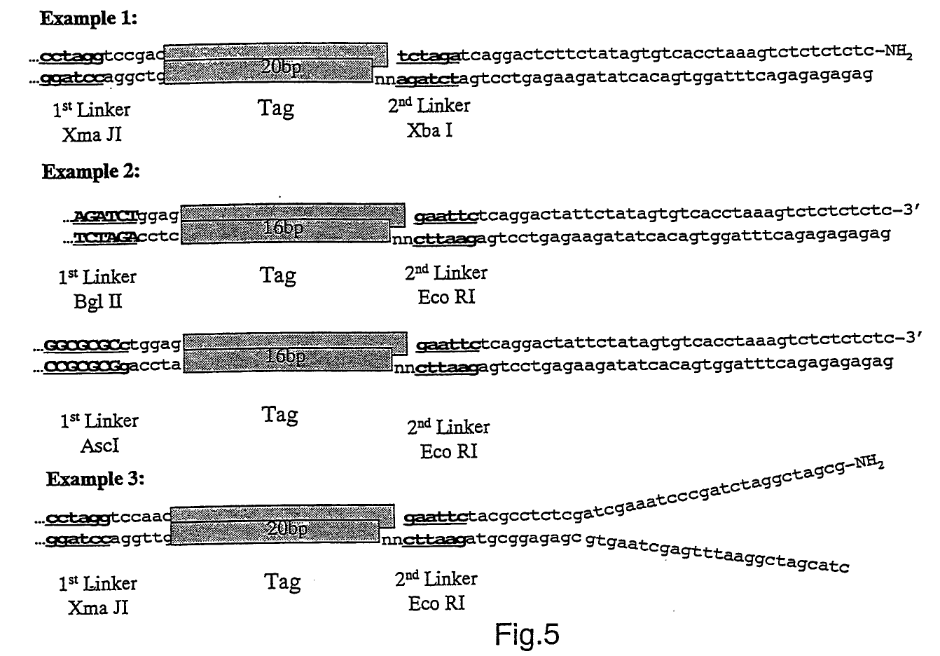

FIG. 6 shows examples for illustrating the structure of a dimer of 5′ end tags prepared in accordance with Examples 1 to 3. Note that in the case of concatemers prepared according to Example 1 different linker sites can be found as XmaJI and XbaI create the same overhangs after digestion, which can be recombined. One example for such a concatemer is given in the figure.

### DETAILED DESCRIPTION OF PREFERRED EMBODIMENTS

As described above, the method of the present invention can comprise, but is not limited to, roughly three steps each of which further comprises a plurality of steps. Each step will now be explained below. The concrete working examples of each step is described in detail in the later-mentioned working examples.

Step 1

Step 1 is to selectively collect cDNAs containing a site corresponding to the 5′ end of mRNAs in a sample. The cDNAs may be synthesized for instance by using said mRNAs as templates.

Either total RNA or mRNA taken from a desired cell, tissue, or organism can be used as the starting substrate. Methods for preparation of total RNA and mRNA are already known, and it is also described in the later-mentioned working examples. Alternatively, a cDNA library itself may be cleaved if it carries a recognition side for a Class IIS or Class III enzyme in proximity of the 5′ end of its inserts.

Also, a full-length cDNA library may be used to isolate the 5′ end nucleic acids corresponding to the 5′ end of the transcribed part of a gene.

Step 1 itself can be conducted by a publicly known method. In other words, methods to construct full-length cDNAs and methods to synthesize cDNA fragments at least containing a site corresponding to the 5′ end of the mRNAs are already known, and any of these methods can be adopted. One of the preferable methods is the cap trapper method (e.g. Piero Carninci et al., Methods in Enzymology, Vol. 303, pp. 1944, 1999). This cap trapper method shall be explained below; however, the invention is not limited to the use of the cap trapper method and other approaches to enrich or select full-length cDNAs could be applied as well.

The cap trapper method first synthesizes the first-strand cDNA with a reverse transcriptase using RNA as a template. This can be conducted by a known method. The cDNA can be primed with an oligo-dT primer or, when the template RNA is mRNA, it can be primed with a random primer. It is advisable to add trehalose to the reactive solution because it raises the efficiency of reverse transcription reaction by stabilizing the reverse transcriptase (U.S. Pat. No. 6,013,488). It is preferable to use 5-methyl-dCTP instead of standard dCTP, because it avoids internal cDNA cleavage with several restriction enzymes and prevents unintended cleavage with restriction enzymes to a considerable extent. In addition, after the first-strand cDNA synthesis, proteins and digested peptides might be removed by CTAB (cetyl trimethyl ammonium bromide) treatment, or other more general methods to purify cDNA.

Next, a selective binding substance is bound to the cap structure of mRNA A “selective binding substance” here means a substance that selectively binds to a specific substance. Such selective binding substance includes preferably biotin, but is not limited to biotin. The cap structure is the structure at the 5′ end of mRNA, but not found in transfer RNA (tRNA) or ribosomal RNA (rRNA), thus allowing for a specific selection of mRNA molecules. Therefore, even if total RNA was used as the starting substrate, the selective binding substance only binds to mRNA. In addition, the selective binding substance does not bind to mRNA if the cap structure at the 5′ end has been lost. Biotin can be bound to the cap structure by a known method. For instance, the cap structure can be biotinylated by first oxidizing the diol group within the cap structure by treating mRNA with an oxidizer such as NaIO₄ and making them react with biotin hydrazide.

Single-strand RNA is cleaved by means such as RNase I treatment. Any other RNase that can cleave single strand RNAs but not cDNA/RNA hybrids or cocktails of RNAses that can cleave various single-strand RNA sequences with various specificities can be used alternatively. In an RNA/cDNA hybrid whose first-strand cDNA has not been extended to the site corresponding to the 5′ end of RNA, the vicinity of the 5′ end of RNA is single-stranded due to its failure to be hybridized with cDNA. Thus, the hybrid is cleaved at the single-stranded part and loses its cap structure through this step. Consequently, this step leaves only those mRNA/cDNA hybrids with cDNA that fully extends to the 5′ end of mRNA to maintain the cap structure.

A matching selective binding substance fixed to a support, which selectively binds to the aforementioned selective binding substance, is prepared. In the present specification, a “matching selective binding substance” means a substance that selectively binds to the aforementioned selective binding substance, which, in the case where the selective binding substance is biotin, would be avidin, streptavidin or a derivative thereof that binds specifically to biotin or its derivatives. The support can favorably be, but is not limited to be, magnetic beads, particularly magnetic porous glass beads. Since magnetic porous glass beads to which streptavidin has been fixed are commercially available, such commercial streptavidin coated magnetic porous glass beads can be used. Similarly other materials such as latex beads, latex magnetic beads, agarose beads, polystyrene beads, sepharose beads or alike could be used instead of porous glass beads. Furthermore, the invention is not limited to the use the biotion-avidin system but other binding substances could be used like a digoxygenin tag that would be attached to the cap structure and digoxygenin recognizing antibodies attached to a solid matrix.

Following this, the aforementioned mRNA/cDNA hybrid with the cap structure is made to react with the aforementioned matching selective binding substance fixed to the support in order to bind the selective binding substance on the cap structure with the matching selective binding substance on the support, thereby immobilizing the mRNA/cDNA hybrid with the cap structure on the support. When magnetic beads are used as the support, applying a magnetic force can quickly collect the magnetic beads. Meanwhile, in order to prevent non-specific binding to the support, it is preferable to treat the support with a large excess of DNA-free tRNA for blocking such binding before conducting this reaction. Other substances that are suitable for blocking the surface are nucleic acids or derivatives, for instance total RNA or oligonucleotides; proteins, for instance bovine serum albumine; polysaccharides, for instance glycogen, dextran sulphate, heparin or other polysaccharides. Hybrid molecules containing parts of all of the above could be used to mask non-specific binding sites.

The above focuses on the case where Step 1 is conducted by the cap trapper method, but other methods can also be used as long as they can selectively collect cDNAs containing a site complementary to the 5′ end of mRNA.

Alternatively to the cap-selection, one could dephosphorylate the 5′ ends of mRNAs with a phosphatase, such as BAP (bacterial alkaline phosphatase), followed by treatment with the decapping enzyme TAP (tobacco acid pyrophosphatase). Subsequently a ribonucleotide or a deoxyribonucleotide can be attached to the 5′ end of the mRNA instead of the original cap-structure with RNA ligase (Maruyama K, Sugano S Gene 138, 171-4 (1994)). In this way, for instance a Class II or Class III recognition site can be placed in the oligonucleotide or ribonucleotide sequence used during the ligation step, which is placed at the 5′ end of a cDNA or RNA. This Class II or Class III restriction enzyme can then be used to cleave within the cDNA and produce the 5′ end tag.

Alternatively to biotin, a cap-binding protein (Pelletier et al. Mol Cell Biol 1995 15:3363-71; Edery I. et al., Mol Cell Biol 1995 June; 15(6):3363-71) or an antibody (Theissen H et al. EMBO J. 1986 Dec. 1; 5(12):3209-17) that specifically binds to the cap structure can be used as the aforementioned selectively binding substance.

Alternatively, one could use methods to attach oligonucleotides chemically to the cap structure as described by Genset. This method is based on the oxidation of cap structure (U.S. Pat. No. 6,022,715). This allows (1) adding to the cap an oligonucleotide which may contain a recognition side for a Class IIS or Class III restriction enzyme, and (2) preparing first-strand cDNA which then switches second-strand cDNA synthesis.

Alternatively, one could use the cap-switch method as described by Clontech (U.S. Pat. No. 5,962,272). One could prepare the first-strand cDNA in presence of a cap-switch oligonucleotide which carries a recognition site for a substance, capable of recognizing nucleic acids and cleaving them apart from the recognition sequence, so that Class IIS or Class III restriction enzyme may be used. The cap switch mechanism lets the first strand synthesis continue on the cap-switch oligonucleotides. This can be continued by a second-strand cDNA synthesis, or followed by a PCR step as describes for instance in the SMART™ Clontech cloning system.

In another embodiment, depending on the quality of RNA, random priming and extending the cDNA up to the cap-structure may allow for the utilization of 5′ ends. Particular enzyme and reaction conditions allow sometimes reaching the cap-site with high efficiency (Carninci et al, Biotechniques, 2002). Even without a cap-selection it is possible to attach, in place of the cap structure, oligonucleotides which carry Class IIS or Class III restriction enzyme sites that would be later used to produce concatemers.

Finally, the cDNA can be cleaved with the Class II (Class IIS or Class IIG) or Class III restriction enzyme to produce 5′ end tags. The 5′ end tags are used in the subsequent formation of concatemers. Any other methods, including mechanical cleavage, may possibly be used.

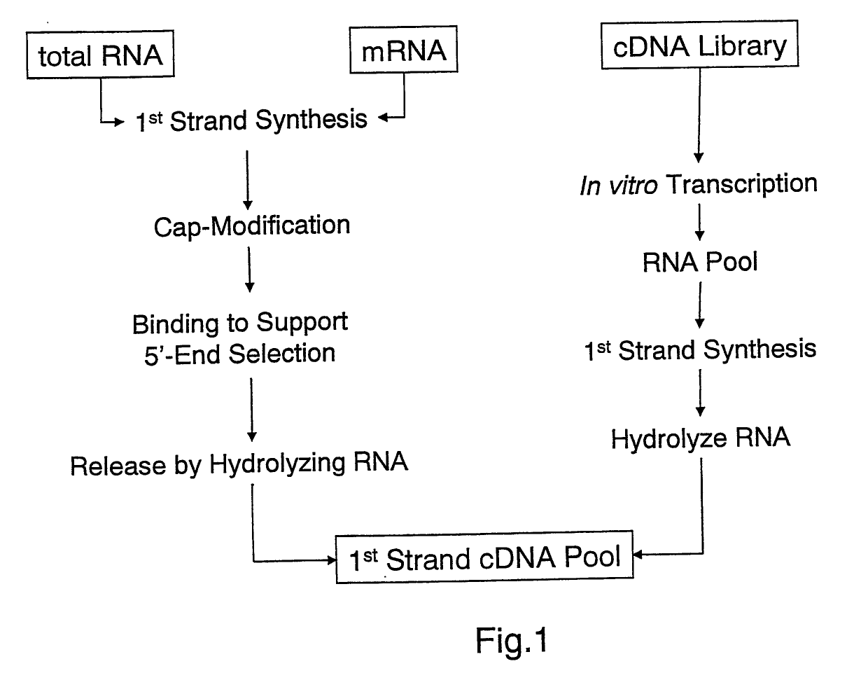

FIG. 1 summarizes expamplary workflows according to the present invention.

According to FIG. 1, to perform the method of the present invention, 5′ ends of transcribed regions can be isolated from a plurality of RNA molecules or total RNAs, a plurality of RNA molecules which have been enriched for mRNA fractions, or a full-length cDNA library.

When applying the present method to a plurality of total RNA or mRNA molecules, mRNA molecules may be used as templates to synthesize complementary cDNA strands. The cDNA strands preceed to a selection step so as to enrich mRNA/cDNA hydrides comprising the 5′ ends of the transcribed regions. After the removal or destruction of the mRNA portion by hydrolysis with an alkali, a first-strand cDNA pool comprising the 5′ ends of the transcribed regions is prepared.

In a different embodiment of the invention, a full-length cDNA library can be used to prepare a RNA pool comprising the 5′ ends of the cDNA clones. A single-stranded cDNA pool is then synthesized using the aforementioned RNA pool as a template. A first-strand cDNA portion thereof is obtained after the removal or destruction of the RNA molecules by hydrolysis with an alkali, and the resulting first-strand cDNA pool comprises the 5′ ends of the transcribed regions. The transcribed regions are available for further processing under the present invention. Note that when starting from a full-length cDNA library no selection for 5′ ends is required.

Step 2

In continuation of Step 1, the following Step 2 is carried out to selectively collect fragments containing a cDNA site that at least contains a site complementary to the 5′ end of mRNA.

When using the aforementioned cap trapper method, the first-strand cDNA that has been immobilized on the support is released. It can be conducted by treating the support with alkali, such as sodium hydroxide. Alternatively to alkali, an enzymatic reaction with RNaseH (which cleaves only the RNA hybridized to DNA) could be used. The alkali treatment releases the cDNA from the mRNA/cDNA hybrid, bound to the support through the cap on the mRNA and separates the cDNA from the mRNA to only leave first-strand cDNA on its own.

Then, a linker is added to the cDNA that holds a sequence recognized in a sequence-specific manner by a substance having an enzymatic activity that cleaves the recognized DNA outside the recognition sequence. Such substances include but are not limited to certain Class II and Class III restriction enzymes.

In this embodiment, a linker that at least carries a Class IIS or Class III restriction enzyme site and a random oligomer part at the 3′ end are ligated to the end of this first-strand cDNA, which corresponds to the 5′ end of the aforementioned mRNA (i.e., the 3′ end of the cDNA). For the later cloning of the 5′ end sequence tags into concatemers, it is preferable, but not essential, to introduce a second recognition site into the linker. The second recognition site should be distinct from the aforementioned recognition site used for, for example, the Class IIS or Class III restriction enzyme.

This can preferably be conducted using a linker that carries a Class IIS or Class III restriction enzyme site and a random oligomer part (SSLLM (single strand linker ligation method), Y. Shibata et al., BioTechniques, Vol. 30, No. 6, pp. 1250-1254, (2001)). The Class IIS and Class III restriction enzymes are restriction enzyme groups that cause cleavage at parts other than the recognition site. An example for a Class IIS restriction enzyme includes, but is not limited to, the use of GsuI. GsuI treatment cleaves one of the strands at 16 bp downstream from the recognition site, and the other strand at 14 bp downstream from the recognition site. Another suitable example is MmeI, which cleaves respectively 20 and 18 bases apart from its recognition sequence. An example for a Class III restriction enzyme includes, but is not limited to, EcoP15I, which cleaves respectively 25 and 27 bp apart from its recognition site. The random oligomer part is located at the 3′ end of the linker, and though the number of bases is not particularly restricted, the recommended number is 5 to 9, or more preferably, 5 to 6. The Class IIS or Class III restriction enzyme site should be located close to the aforementioned random oligomer par; so that the cleavage point comes within the cDNA The linker should preferably be a linker of double-stranded DNA of which the aforementioned random oligomer part protrudes to the 3′ end and provides the binding end. In addition, it is advisable to bind a selective binding substance such as biotin to the linker in advance to facilitate its collection later.

When the aforementioned first-strand cDNA is made to react with such a linker, the random oligomer part of the linker hybridizes with the 3′ end of the first-strand cDNA (i.e. the 5′ end of the template mRNA). Next, the second-strand cDNA is synthesized by using this linker as a primer and the first-strand cDNA as a template. This step can be conducted by a standard method. In a different embodiment of the invention, the first-strand cDNA can be subtracted by hybridization against a plurality nucleic acids followed by physical separation of single-stranded and double-stranded DNA-DNA or DNA-RNA hybrids. Such a subtraction step can be performed by, but is not limited to, the method disclosed in U.S. patent publication No. 20020106666. Single-stranded cDNA retrieved from the subtraction step is used as a template for second strand synthesis by standard procedures similar to the aforementioned approach omitting a subtraction step.

Then, the obtained double-strand cDNA is treated with the above Class IIS or Class III restriction enzyme. In this step, a double-strand cDNA fragment comprising a linker-derived part and a part derived from the 5′ end of the cDNA (the 5′ end of the second-strand cDNA) is prepared. For instance, if GsuI is to be used as the Class IIS restriction enzyme and if a linker is designed to locate the restriction site immediately upstream from the aforementioned random oligomer site, the obtained DNA fragment would include a site derived from the site on the 5′ end of the second-strand DNA (i.e. the site on the 5′ end of the mRNA) of the length of 16 bp (however, the complementary strand is 14 bp). In the case of using Mme I, the length of the second-strand DNA fragment should increase to 20 and 18 bp, respectively, and in the case of EcoP15I to 25 and 27 bp, respectively.

Next, such DNA fragments are selectively collected. If a selective binding substance (e.g. biotin) had been bound to the linker as above, the collection could be conducted similarly to Step 1 by using a support to which a matching selective binding substance (e.g. streptavidin) would be fixed. This procedure completes Step 2, which selectively collects fragments containing a cDNA site, belonging to the first-strand cDNA, which at least contains a site complementary to the 5′ end of the aforementioned mRNA.

The above explains the case where the SSLLM is used for Step 2, but Step 2 can also be carried out by any other method as long as the method can selectively collect fragments containing the 3′ end of the first-strand cDNA (the 5′ end of the template mRNA). For instance, it is possible to use exonuclease that cleaves the nucleotide in the 5′ to 3′ direction at a controlled speed. The exonuclease treatment of the first-strand cDNA for a prescribed time period leaves a single-strand fragment comprising the 3′ end of the first-strand cDNA (the 5′ end of the template mRNA). It is possible to obtain only the targeted single-strand fragments by conducting treatment with a nuclease that only splits double-strand fragments. These fragments can be collected, joined with adapters and cloned.

The above selected fragments that correspond to the 5′ end can be further ligated to linkers and then used for PCR amplification in case that the quantity is insufficient for the downstream applications such as cloning.

In one embodiment, the fragments corresponding to the 5′ part of mRNAs is ligated on the 3′ end to a linker carrying just another restriction enzyme site, which may be distinct from the restrictions site used in the first linker. Thereafter, the fragments corresponding to the 5′ end of mRNA contain linkers carrying recognition sites for restriction enzymes at both sides. Such fragments can be amplified by PCR followed by subsequent cleavage by one or two restriction enzymes to produce DNA fragments suitable for the cloning of concatemers as described below in more detail.

In another embodiment similar to (Velculescu et al, 1995), the aforementioned DNA fragment or PCR product is initially used for forming dimmeric molecules comprised of two 5′ end specific fragments ligated to one another in opposite orientation. These dimmers can then be used directly or after just another PCR amplification to produce concatemers as specified in more detail below.

In just another embodiment of the invention, alternatively to PCR amplification DNA RNA polymerase could linearly amplify fragments corresponding to 5′ ends having appropriate linkers at both ends. DNA fragments are then reconstituted by a reverse transcription step and a second strand formation to allow for concatemer formation.

Step 3

The subsequent Step 3 forms concatemers by mutually ligating the collected fragments. Since there are multiple mRNAs and the linker hybridizes with the first-strand cDNA at the random oligomer part as above, the above method can obtain fragments containing multiple cDNAs derived from multiple mRNAs within a sample. Step 3 ligates these multiple fragments and forms concatemers. The ligation of the cDNA fragments can be carried out by a standard method, using commercial ligation kits based on but not limited to T4 DNA ligase. The ligation can be securely conducted but is not limited to a method, which first is introducing a second linker providing a recognition site for a restriction enzyme that is distinct from the other recognition sites used at the earlier stages, which is then ligating two fragments into dimmers comprising two 5′ tags in the opposite direction (di-tag), and which is further ligating such ligated di-tag fragments into concatemers as described in more detail in Example 2 and 3. However, the performance of the invention is not dependent on the cloning of intermediary di-tags. As described in more detail in Example 1, monomeric tags can be self-ligated directly to form concatemers of satisfying length to perform the invention. Thus the invention is neither limited to nor dependent on the use of di-tags. The number of ligated fragments is not restricted, practically any number above two and preferably at least 20-30 is suitable to perform the invention. The obtained concatemers are preferably but not limited to be amplified or cloned by a standard method.

The concatemers obtained in this way each comprise a site having the same base sequence (however, uracil in RNA would be thymine in DNA) as that of the 5′ end of the multiple mRNAs within the sample. Although it also comprises a part derived from the linker or linkers, the base sequence of the linker or linkers is known from the experimental design, so the part derived from the linker or linkers and the part derived from mRNA can be clearly distinguished by investigating the base sequence of the concatemer. Therefore, by determining the base sequence of the obtained concatemer, it is possible to find out the base sequences at the 5′ end of multiple mRNAs within the sample. The base sequences of a maximum of 16, 20 or 25 bases at the 5′ end of each mRNA can be learned by the preferable mode of using GsuI, Mme I or EcoP15I. Information on 16, 20 or 25 bases would be sufficient for almost definitely identifying the mRNA statistically and to judge whether or not it is a new mRNA. In addition, by determining the base sequence of the concatemer, it is possible to learn the base sequences at the 5′ end of mRNAs for the number of above fragments included in the concatemer (preferably 20 to 30), so information on the 5′ end of multiple mRNAs can be determined efficiently. The analysis of the concatemers can be automated by the use of computer software to distinguish between sequences derived forms the 5′ ends and sequences derived from a linker or the linkers.

Sequences from specific 5′ end tags obtained from concatemers in the aforementioned form can be analyzed for their identity by standard software solutions to perform sequence alignments like NCBI BLAST (http://www.ncbi.nlm.nih.gov/BLAST/), FASTA, available in the Genetics Computer Group (GCG) package from Accelrys Inc. (http://www.accelrys.com/), or alike. Such software solutions allow for an alignment of 5′ end specific sequence tags among one another to identify unique or non-redundant tags for clustering and further use in database searches. All such non-redundant sequence tags can then be individually counted and further analyzed for the contribution of each non-redundant tag to the total number of all tags obtained from the same sample. The contribution of an individual tag to the total number of all tags should allow for a quantification of the transcripts within a plurality of mRNAs or a cDNA library. The results obtained in such a way on individual samples can be further compared with similar data obtained from other samples to compare their expression patterns against each other. Thus the invention allows for the expression profiling of individual transcripts within one or more samples and the establishment of a reference database.

Specific 5′ end sequence tags obtained as describe above can further be used to identify transcribed regions within genomes for which partial or entire sequences were obtained. Such a search can be performed using standard software solutions like NCBI BLAST (http://www.ncbi.nlm.nih.gov/BLAST/) to align the 5′ end specific sequence tags to genomic sequences. Though 20 bp tags were found to map specifically to genomic sequences, in some cases it may be necessary to extend the initial sequence information obtained from concatemers for example by one of the approaches described below. The use of extended sequences allows for a more precise identification of actively transcribed regions in the genome. Similarly, the same approach and software solutions can be used to search for related sequences in other databases e.g. like NCBI (http://www.ncbi.nlm.nih.gov/Database/index.html), EMBL-EBI (http://www.ebi.ac.uk/Databases/index.html), or DNA Data Bank of Japan (http://www.ddbj.nig.ac.jp/).

Specific 5′ end sequence tags which could be mapped to genomic sequences allow for the identification of regulatory sequences (Suzuki Y et al. EMBO Rep. 2001 May; 2(5):388-93 and Suzuki Y et al. Genome Res. 2001 May; 11(5):677-84). In a gene the DNA upstream of the 5′ end of transcribed regions usually encompasses most of the regulatory elements which are used in the control of gene expression. These regulatory sequences can be further analyzed for their functionality by searches in databases which hold information on binding sites for transcription factors. Publicly available databases on transcription factor binding sites and for promoter analysis including Transcription Regulatory Region Database (TRRD) (http://wwwmgs.bionet.nsc.ru/mgs/dbases/trrd4/), TRANSFAC (http://transfac.gbf.de/TRANSFAC/), TFSEARCH (http://www.cbrc.jp/research/db/TFSEARCH.html), and PromoterInspector provide by Genomatix Software (http://www.genomatix.de/) provide resources for computational analysis of promoter regions.

Sequence information obtained from 5′ end specific sequence tags or obtained by mapping a 5′ end sequences to a genome can be further used to manipulate the regulation of a given target gene. In such an experiment promoter related information would be used to alter its activity or to replace it with an artificial promoter. Alternatively, 5′ end specific tags could provide sequence information for the design of anti-sense or RNAi probes for gene inactivation.

In a different embodiment of the invention, sequence information derived from the concatemers can be used to synthesize specific primers for the cloning of full-length cDNAs. In such an approach, the sequence derived from a given 5′ end specific tag is used to design a forward primer while the choice of the reverse primer would be dependent on the template DNA used in the amplification reaction. Amplification by the polymerase chain reaction (PCR) can be performed using a template derived from a plurality of RNA obtained from a biological sample and an oligo-dT primer. In the first step the oligo-dT primer and a reverse transcriptase are used to synthesize a cDNA pool. In the second step a forward primer derived from a 5′ end specific tag and an oligo-dT primer are used to amplify a full-length cDNA from the cDNA pool. Similarly, a specific full-length cDNA can be amplified from an excisting cDNA library using a forward primer derived from a 5′ end tag and a vector nested reversed primer.

While the above method had used mRNA or total RNA within the sample as the starting substrate, Step 1 can be omitted by using an existing full-length cDNA library. In this way, information on the base sequences of the 5′ end of multiple cDNAs (i.e. the 5′ end of the mRNAs used as templates for said cDNAs) contained in the full-length cDNA library can be efficiently obtained similarly to the above procedure.

Independent from the starting material used to perform the invention, the single-stranded first-strand cDNA material can be fractionated by means of subtractive hybridizations and physical separation to allow for enrichment of 5′ ends of differentially expressed genes or for the concentration of transcripts of low abundance.

In some embodiments it could be desirable to obtain extended sequence information from the 5′ ends of transcribed regions. Such extended sequences may allow in specific cases for the identification of start sites of protein synthesis or a better mapping to genomic sequences. As described above the invention included in Step 2 the ligation of a linker to the 5′ end of a cDNA. Introducing a single-stranded overhang encompassing a sequence obtained from a concatemer to bind to and to be ligation to a specific nucleic acid fragment such a linker can used in a target specific manner. After the ligation the linker can be used to enrich the DNA fragment by attaching the linker to a support from which it could be released after the enrichment. The linker can further be used as a primer to obtain extended sequence information on 5′ ends in a liquid phase or on the solid phase used before for enrichment.

By investigating the base sequences of the concatemers or extended 5′ sequences obtained by the present invention, it is not only possible to clone new genes as described above, but also possible to investigate the expression profiles of genes within the sample. Furthermore, the technology can be used for various purposes such as to map transcription start sites in the genome, to map promoter usage patterns, for the analysis of SNPs in promoter regions, for creating gene networks by combining the expression analysis with information on promoters, alternative promoter usage and on availability of transcription factors, and for selective collection of the promoter site within fragmented genomic DNA. To select genomic fragments containing promoter sites, a fragment containing the same base sequence as the 5′ end of mRNA could be bounded to a support e.g. by using the aforementioned biotin system, and hybridized to fragmented genomic DNA. Hybridized genomic DNA fragments could then be separated from a mixture of genomic fragments by using e.g. streptavidin coated magnetic beads, and cloned under standard conditions.

Alternatively, concatemer cloning could be avoided by making and using selected 5′ end tags ligated to a mixture of full-length cDNAs and bound to magnetic beads carrying homogeneous sequence of oligonucleotides, followed by ligation such as in the SSLLM, second-strand cDNA preparation and cleavage with a Class IIS or Class III restriction enzyme. The 5′ end specific tag would be anchored specifically to the beads and would be used for the specific sequencing similarly as done by Lynx Therapeutics (U.S. Pat. Nos. 6,352,828; 6,306,597; 6,280,935; 6,265,163; and 5,695,934).

For instance, oligonucleotides would have a “random part I”, which will bind to 5′ ends of cDNAs; and a code part of the oligonucleotide, which will be able to “tag” the ligation product. The oligonucleotide may be destroyed by exonuclease VII if not hybridized with a cDNA. The “decoder” oligonucleotides would be used to select out the sequence. The specific arrays of cDNAs on beads are then arrayed onto a solid surface, one per position, followed by parallel sequencing. The aforementioned approach would allow for the design of a liquid array format, in which each bead could be addressed by an independent label and processed individually for sequence analysis or alike.

In a different embodiment of the invention known 5′ end specific tags can be used for an alternative analysis of 5′ end specific sequences omitting the cloning and sequencing of concatemers. In such a case 5′ end specific oligonucleotides of about 25 bp would be synthesized and fixed to a solid support to form a 5′ end specific microarray. The hybridization of 5′ tags obtained from a sample would then allow for the identification and quantification transcripts present in the sample. Standard methods for the preparation and use of microarrays are know to a person trained in the state of the art of molecular biology (Jordan B., DNA Microarrays: Gene Expression Applications, Springer-Verlag, Berlin Heidelberg New York, 2001: Schena A, DNA Microarrays, A Practical Approach, Oxford University Press, Oxford 1999).

By modifications as the aforementioned approaches for direct sequencing of 5′ ends or a readout by hybridization to a 5′ end specific microarray the invention provides different means for the general analysis of 5′ ends in the form of concatemers or the analysis of individual 5′ ends, which were enriched by means of a 5′ end specific selection.

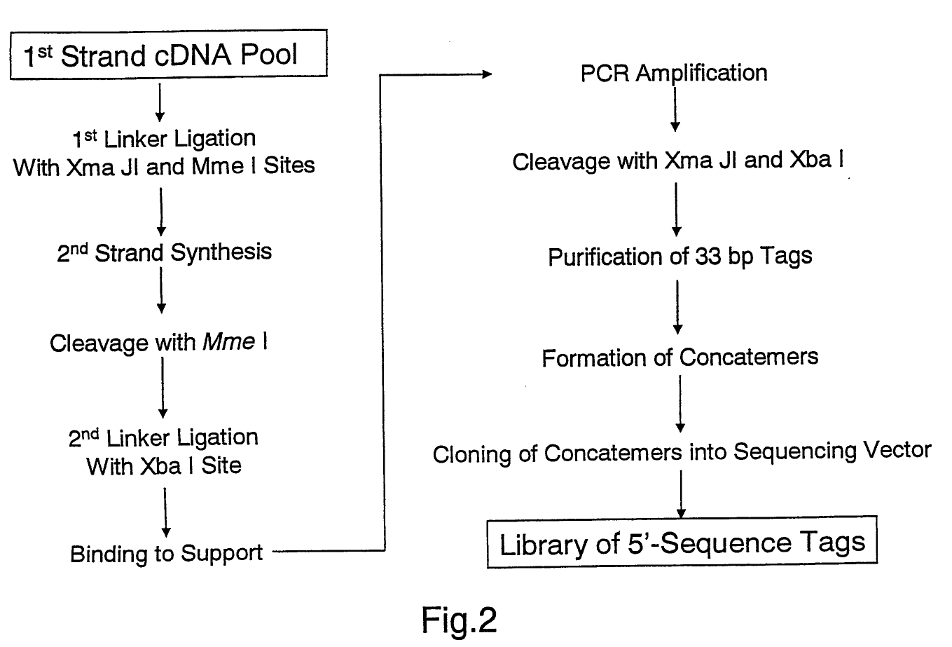

FIG. 2 summarizes the exemplary work flow according to Steps 2 and 3 discussed above.

In FIG. 2, the restriction enzymes Xma JI, Mme I and Xba I are used for the cloning of 33 bp DNA fragments as described in more detail in the Example 1 below. In principle, the cloning of 5′ end specific tags comprises the following steps.

In the initial step of the invention outlined in FIG. 1, a pool of single-stranded cDNA is obtained. The pool comprises the 5′ end regions transcribed from the mRNAs. Adjacent to the portion of the single-stranded cDNA which contains the 5′ end regions transcribed from the mRNAs, a specific linker, here denoted as “1^{st} Linker”, is ligated to provide a recognition site for a restriction enzyme that cleaves outside the lt linker with respect to its binding site or within the 5′ end transcripeted region. For the purpose of the example described in the figure, the restriction enzyme Mme I is used as it cleaves 21 bp downstream of the recognition site, thus allowing for the termination of tags which comprise the 5′ ends of transcribed regions of mRNAs. Also, a second restriction enzyme is given for the “1^{st} Linker.” For the purpose of this example, Xma JI is used for the later cloning of the 5′ end specific tags.

Subsequently, the “1^{st} Linker” is used to prime the synthesis of a second complementary cDNA strand, resulting in double-stranded cDNA molecules which comprise the 5′ ends of transcribed regions of the mRNAs and which have a recognition site for restriction enzymes that cleave at a site located outside the 1^{st} Linker with respect to its binding site adjacent to the region containing the 5′ end regions transcribed the mRNAs.

The aforementioned restriction enzyme that cleaves the outside of the binding site is, for the purpose of this example, Mme I. Cleavage with Mme I results in double-stranded cDNA fragments of the tags which comprise the 5′ ends of transcribed regions of the mRNAs and the “1^{st} Linker” and which have a single strand DNA overhang at the cleavage site of Mie I.

To the aforementioned single-stranded DNA overhang at the cleavage site of Mme I, a “2^{nd} Linker” is ligated to provide a recognition site for a restriction enzyme suitable for the cloning of the cDNA fragments or tags which function as templates for amplification by means of PCR.

The cDNA fraction comprising the “1^{st} Linker”, cDNA fragments comprising the 5′ ends of regions transcribed from the mRNAs, and the “2^{nd} Linker” is purified by selective binding to a support by the means of a selective binding substance attached to the 1^{st} Linker.

For the purpose of the cloning of the cDNA fragments comprising the 5′ ends of transcribed regions or tags, the aforementioned cDNA fraction comprising the “1^{st} Linker”, cDNA fragments or tags which comprise the 5′ end regions transcribed from mRNA, and the “2^{nd} Linker” are amplified by means of PCR, and the linker portions are cleaved off by restriction enzymes to allow for the ligation of the tags into concatemers. For the purpose of this example, the restriction enzymes Xma JI and Xba I are used, which cleave out a 33 bp fragment from the aforementioned cDNA fragments. After an appropriate purification step, the 33 bp fragments are ligated to each other for the formation of concatemers comprising, for example, up to 30 tags comprising the 5′ ends of transcribed regions said mRNA or cloned individually.

The concatemers can be cloned into a sequencing vector to prepare a library comprising the 5′ end regions transcribed from mRNA.

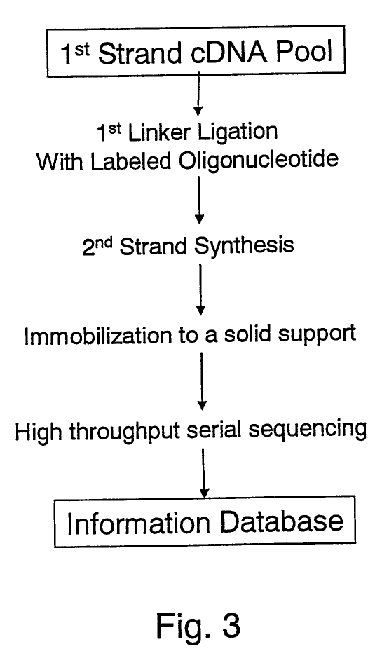

FIG. 3 shows a principle workflow according to the present invention to illustrate an alternative approach for the direct sequencing of 5′ end tags. For the purpose of this embodiment of the invention, the single-stranded cDNAs which comprises the 5′ end regions transcribed from the mRNAs and obtained as summarized in FIG. 1 are ligated to a linker, here denoted as “1 Linker”, which for the purpose of this example, has a specific label to allow for the immobilization of the ligation product on a solid support. This linker can be used as a primer for the synthesis of a 2^{nd} strand cDNA complementary to the first strand. The single-stranded DNAs having a double-stranded linker adjacent to the region comprising the 5′ end regions transcribed from the mRNAs or double-stranded DNA comprising the 5′ end transcribed regions can be forwarded for individual or parallel sequencing, for the purpose of this example; by a high throughput serial sequencing approach for the 5′ ends of mRNAs.

The present invention will now be described by way of examples thereof. It should be noted that the present invention is not restricted to the Examples. The experiments described in the Examples can be performed by any person experienced in the state of the art of standard techniques in the field of Molecular Biology. Unless otherwise defined in the text, the technical terms, abbreviations, and solutions used in the Examples should have the same meaning as commonly understood by a person experienced to the state of the art in the field of the invention. A general description of such terms, abbreviations and solutions can be found in the common reagent section in Molecular Cloning (Sambrook and Russel, 2001). All publications mentioned herein are incorporated into this document by reference to be disclosed and to describe the methods and/or materials therein.

### EXAMPLES

### Example 1

### Preparation of 5′ End Specific Tags According to the Invention Omitting Di-Tags

To perform the invention mRNA or total RNA samples can be prepared by standard methods known to a person trained in the art of molecular biology as for example given in more detail in Sambrook and Russel, 2001. Carninci P. et al. (Biotechniques 33,306-9, (2002)) described one such method used herein to obtain cytoplasmic mRNA fractions, however, the invention is not limited to this method and any other approach for the preparation of mRNA or total RNA should allow for the performance of the invention in a similar manner.

The preparation of mRNA from total RNA or cytoplasmic RNA is preferable but not essential to perform the invention as the use of total RNA can provide satisfying results in combination with the cap-selection step described below in this example. Generally speaking, mRNA represents about 1-3% of the total RNA preparations, and it can be subsequently prepared by using commercial kits based on oligo dT-cellulose matrixes. Such commercial kits including, but not limited to, the MACS mRNA isolation kit (Milteny) provided satisfactory mRNA yields under the recommended conditions when applied for the preparation of mRNA fractions for performing the invention. To perform the invention one cycle of oligo-dT mRNA selection is sufficient as extensive mRNA purification can particularly cause the lost of long mRNAs.

All mRNA samples used to perform the invention were analyzed for their ratios of the OD readings at 230, 260 and 280 nm to monitor the mRNA purity. Removal of polysaccharides was considered successful when the 230/260 ratio was lower than 0.5 and an effective removal of proteins was obtained when the 260/280 ratio was higher than 1.8 or around 2.0 The RNA samples were further analyzed by electrophoresis in an agarose gel and to prove a good ratio between the 28S and 18S rRNA in total RNA preparations.

The first-strand cDNA was prepared from different mRNA samples using Superscript II (Invitrogen) under the following conditions:

In a final volume of 22 μl 5-25 μg of purified mRNA or up to 50 μg of total RNA were mixed with 14 μg of the appropriate purified 1^{st} strand cDNA primer (5′-(GA)₅AAGGATCCTGCCATTTCATTACCTCTTTCTCCGCACCCGACATAGA(T)₁₆VN-3′) (SEQ ID NO: 1) and heated to 65° C. for 10 min to allow for annealing of the primer and afterwards immediately placed on ice.

In a second tube the reaction mixture for the first-strand synthesis was prepared with a final volume of 128 μl:

| | | |
|---|---|---|
| 2× GC I (LA Taq) buffer (TaKaRa) | 75 | μl |
| dATP, dTTP, dGTP, and 5-methyl-dCTP, 10 mM each | 4 | μl |
| 4.9 M sorbitol | 20 | μl |
| Saturated trehalose (approximately 80%) | 10 | μl |
| Superscript II reverse transcriptase (200 U/μl) | 15 | μl |
| ddH₂O | 4 | μl |

A third reaction tube with 1.5 μl of α²P-dGTP (Amersham Pharmacia Biosciences BioTech) was prepared, and the reaction mixture along with the reaction tube holding the radioactive tracer and the RNA template were heated to 42° C. When all solutions had reached the starting temperature of 42° C. the reaction mixture and the RNA template were mixed quickly and out of this solution 40 μl were transferred into the reaction tube holding the radioactive tracer. The remaining reaction mixture with the RNA can be processed in parallel with the radioactive reaction mixture. The first-strand cDNA synthesis was performed in a thermocycler with the following settings: 42° C. for 30 min; 50° C. for 10 min; and 55° C. for 10 min. After having concluded the cycle the reaction was stopped by adding EDTA solution (from a stock of 0.5M) to a final concentration of 10 mM. It is not essential for the performance of the invention to include a radioactive tracer during the first-strand cDNA synthesis, though it can be very helpful to measure the synthesis rate of the reaction and to analyze the cDNA e.g. by alkali gel electrophoresis. Radioactive and non-radioactive materials can be mixed in a new tube and processed together for the following steps. Adding protease K to a final concentration of 1 μg/μl destroyed remaining enzyme activity in the reaction mixture after an incubation at 50° C. for 15 min or longer. From the reaction mixture RNA and first-strand cDNA were isolated by precipitation with CTAB urea followed by ethanol as described below. To a reaction mixture of about 128 to 142 μl, 32 μl of 5 M sodium chloride and 320 μl of a 1% CTAB (cetyl trimethyl ammonium bromide) solution in 4M urea were added and mixed carefully. The solution was incubated at room temperature for 10 min before the precipitate was isolated by centrifugation at 15,000 rpm for 10 min. The supernatant was removed and the pellet carefully re-suspended in 100 μl of 7M guanidine chloride. For the ethanol precipitation 250 μl of absolute ethanol were added and the mixture and left at −80° C. for 60 min to allow for the formation of the precipitate. The precipitate was collected by centrifugation at 15,000 for 10 min and subsequently washed twice with 800 μl of 80% ethanol. Finally the pellet was re-suspended in 46 μl of water.

In the example described here the invention made used of the so-called cap trapper method for full-length cDNA selection. As the invention is not limited in its performance to the cap trapper method other means for full-length selection can be applied in a similar way. The cap trapper selection was initiated by biotinylation of the cap structure at the 5′ end of mRNA molecules. To the aforementioned first-strand cDNA solution 3.3 μl of 1 M sodium acetate buffer, pH 4.5, and freshly prepared 10 mM NaIO₄ solution, to final concentration of 1 mM, were added and the volume was brought up to a final volume of 55 μL. The mixture was incubated on ice and in darkness for 45 min, and the reaction was then quenched by the addition of 1 μl of 80% glycerol. Out of the reaction mixture RNA and cDNA were isolated by precipitation with isopropanol. To aforementioned reaction mixture, 0.5 μl of 10% SDS, 11 μl of 5M sodium chloride and 61 μl of isopropanol were added, mixed carefully and incubated at −80° C. for 30 min in total darkness. After collecting the precipitate by centrifugation for 15 min at 15,000 rpm, the pellet was washed twice with 500 μl of 80% ethanol. The pellet was finally re-suspended in 50 μL of water. The oxidized diol groups in the mRNA were used to introduce biotin moistures in a reaction with biotin hydrazide. To the aforementioned 50 μl RNA/cDNA solution 160 μl of biotin hydrazide long arm (Vector Laboratories) dissolved at 10 mM concentration in a reaction buffer containing 50 mM sodium citrate buffer pH 6.1, and 0.1% W/V SDS were added to a final volume of 210 μl. The reaction was performed overnight at room temperature to allow for a complete modification of all oxidized diol groups. The reaction was terminated by the precipitation of the RNA and cDNA, for which 75 μl of 1 M sodium citrate, pH 6.1, 5 μl of 5 M sodium chloride and 750 μl of absolute ethanol were added to the reaction mixture. After incubation for 1 h at −80° C. the precipitate was collected by centrifugation at 15,000 rpm for 10 min. The resulting pellet was washed twice with 500 μl of 80% ethanol and finally re-suspended in 175 μl TE buffer (1 mM Tris, pH 7.5, 0.1 mM EDTA).

Full-length cDNAs were further processed from the aforementioned solution by the addition of 20 μl RNase I buffer (Promega) and 1 units of RNase I (Promega, 5 or 10 U/μl) per each 1 μg of starting mRNA or total RNA. The reaction mixture with a final volume of 200 μL was incubated at 37° C. for 30 min before the reaction was stopped by the addition of 4 μl of a 10% SDS solution and 3 μl of a 10 μg/μl proteinase K solution. To destroy the RNase I the reaction mixture was further incubated at 45° C. for additional 15 min. The reaction mixture was then extracted once with 1:1 Tris (pH 7.5)-equilibrated phenol:chloroform before the precipitation of the RNA and DNA. For an improved yield of the precipitation 20 μg of carrier tRNA and 1 volume of isopropanol were added to the reaction mixture and incubated at −20° C. The precipitate was collected by centrifugation at 15,000 rpm for 10 min, washed with 500 μl of 80% ethanol and finally re-suspended in 20 μl of 0.1×TE buffer.

For the isolation of full-length cDNAs magnetic beads coated with streptavidin were used in this example. However, the invention is not limited to the use of magnetic beads as any other solid phase coated with streptavidin or avidin could be used in a similar fashion. To minimize the non-specific binding of nucleic acids to the surface of the magnetic beads, these were pre-incubated before use with DNA-free tRNA. To about 500 μl of magnetic beads slurry (MPG particle, CPG, New Jersey) about 100 μg of tRNA in 10 μl of water was added and incubated on ice for some 30 min with occasional mixing. The magnetic beads were separated from the solution by applying a magnetic force for about 3 min. After the supernatant was removed the beads were washed three times with 500 μl of a binding buffer containing 4.5 M sodium chloride and 0.05 M EDTA to remove free streptavidin from the solution. The beads were then re-suspended in 500 μl of the binding buffer, and out of those 350 μl of the slurry were mixed with the aforementioned RNase-treated cDNA. The resulting slurry was incubated under ongoing agitation at 50° C. for 10 min before adding additional 150 μl of the streptavidin coated magnetic beads. The resulting slurry was again incubated under ongoing agitation for another 20 min at 50° C. Biotinylated full-length mRNA/cDNA hybrids were retained on the magnetic beads and separated from the supernatant by applying a magnetic force. In doing so the beads were washed carefully twice with 500 μl of the binding buffer, once with 500 μl of 0.3 M sodium chloride containing 1 mM EDTA, and finally twice with 500 μl of a buffer containing 0.4% SDS, 0.5 M sodium acetate, 20 mM Tris-HCl pH 8.5, and 1 mM EDTA. Single-stranded cDNAs were released from the beads by alkali treatment of mRNA/DNA hybrids by applying 100 μl of 50 mM sodium hydroxide containing 5 mM EDTA and 5 min incubation at room temperature. During this incubation time the slurry was occasionally mixed. The supernatant was removed and the elution was repeated twice under the same conditions. All three supernatants were pooled and placed on ice immediately. The eluted fractions, about 150 μl, were neutralized by addition of 50 μl of 100 mM Tris pH 8.0, followed by phenol/chloroform extraction and precipitation. The resulting solution of about 200 μl was then treated with RNase I and proteinase K as described above, extracted once with the same volume of Tris-equilibrated phenol:chloroform (ratio 1:1) and out of the aqueous phase the DNA was precipitated with ethanol by adding to 250 μl sample 12.5 μl of 5M sodium chloride, 3.5 μl of 1 μg/μl glycogen, and 250 μl of isopropanol. After incubation at −80° C. for some 30 min, the DNA was collected by centrifugation at 15,000 rpm for 20 min. After having washed the pellet twice with 500 μl of 80% ethanol, the DNA was finally re-suspended in 5 μl of 0.1×TE buffer.

For the next step described in this example a specific linker having a recognition site for the Class IIS restriction enzyme Mme I along with recognition sites for the restriction enzymes XhoI, I-CeuI, and XmaJI was designed. However, the invention is not limited to the use of the restriction enzymes given in this example, and the use of other enzymes is described later in yet a different example. The double-stranded linker was assembled out of two upper strand oligonucleotides with random overhangs and a shorter lower strand oligonucleotide. Note that for the upper strand oligonucleotides, a 4:1 mixture of two oligonucleotides with distinct overhangs was used. The oligonucleotides named below were obtained from Invitrogen Japan and gel purified before annealing. The different end-modifications of the oligonucleotides are indicated below, where “Bio” stands for 5′ biotinylated “Pi” stands for 5′ phosphorylated, and “NH₂” stands for 3′ amino group. The same abbreviations will be used later in the text for other oligonucleotides:

| | |
|---|---|
| Upper oligonucleotide GN5: Bio- | |
| agagagagacctcgagtaactataacggtcctaaggtagcgacctaggtccgacgNNNNN | (SEQ ID NO: 2) |
| Upper oligonucleotide N6: Bio- | |
| ggggacctcgagtaactataacggtcctaaggtagcgacctaggtccgacNNNNNN | (SEQ ID NO: 3) |
| Lower oligonucleotide: | |
| Pi-gtcggacctaggtcgctaccttaggaccgttatagttactcgaggtctctctct-NH₂ | (SEQ ID NO: 4) |

The oligonucleotides were mixed at a ratio of 4×GN5:1×N6:5× “Lower” at a concentration of 2 μg/μl in 100 mM sodium chloride. For annealing the mixture was incubated at 65° C. followed by additional incubations at 45° C. for 5 min, at 37° C. for 10 min, and at 25° C. for 10 min. For ligation of the linker to the single-stranded cDNA 2 μg of linker per 1 μg cDNA were used.

In a final volume of 7.5 μl of 0.1×TE the aforementioned cDNA and the aforementioned linker were mixed and incubated at 65° C. for 5 min to melt secondary structures in the cDNA. The double-stranded linker was then ligated to the single-stranded cDNA using a TaKaRa ligation kit, version 2. Out of the kit 7.5 μl of “Solution II” and 15 μl of “Solution I” were added to the aforementioned annealing reaction mixture, mixed and incubated for 10 h at 16° C. The ligation reaction was terminated by adding 1 μl of 0.5 M EDTA, 1 μl of 10% SDS, 1 μl of 10 mg/ml proteinase K, and 10 μl of water. After incubation at 45° C. for 15 min the resulting mixture was extracted with the three-fold excess of Tris-equilibrated phenol/chloroform. The remaining excess of free linker was removed from the reaction mixture by gel filtrating of the solution in a S-300 spin column (Amersham Pharmacia Biosciences) according to the description of the maker. Briefly, the S-300 columns were transferred into a centrifugation tube and spun at 3,000 rpm for 1 min to remove the storage buffer from the column. After placing the column in a new-centrifugation tube the DNA sample (about 60 μl) followed by another 40 μl of water were added to the column and the column was spun with 3,000 rpm for 5 min at 4° C. to collect the run through. To concentrate the DNA the eluat from the S300 column was placed on a Microcon 100 membrane (Amicon) and centrifuged until a final volume of 10 μl was achieved. The membrane was washed once with 10 μl of 0.1×TE at 65° C. for 3 min and the fractions were united for use in the following second strand synthesis.

For the second-strand cDNA synthesis a thermostable DNA polymerase was applied. As this reaction was performed at a high temperature an excess of upper primer was added to the reaction mixture. This primer was obtained from Invitrogen Japan and gel purified before use. The sequence of the primer resembles the features described above for the upper primer, though no random overhang was included: 5′-Bio-agagagagacctcgagtaactataacggtcctaaggtagcgacctaggtccgacg (SEQ ID NO: 5).

The reaction mixture was set up by mixing the following components:

| | | |
|---|---|---|
| cDNA sample | 10 | μl |
| 100 ng/μl second-strand primer | 6 | μl |
| 5× A buffer (NEB) | 7.2 | μl |
| 5× B buffer (NEB) | 4.8 | μl |
| 2.5 mM dNTP's (Takara) | 6 | μl |
| ddH₂O | up to 45 | μl |

The reaction mixture was heated to 65° C. before 15 μl of 1 U/μl ELONGASE (Invitrogen) were added, and reaction was performed in a thermocycler with the following settings: 5 min at 65° C., 30 min at 68° C., and 10 min at 72° C. The polymerase reaction was terminated by adding 1 μl of 0.5 M EDTA, 1 μl of 10% SDS, and 1 μl of 10 mg/ml proteinase K After incubation at 45° C. for 15 min the resulting mixture was extracted with the same volume of Tris-equilibrated phenol/chloroform (ratio 1:1). The remaining excess of free primer was removed from the reaction mixture by gel filtrating of the solution in an S-300 spin column (Amersham Pharmacia Biosciences) according to the description of the maker. Briefly, the S-300 columns were transferred into a centrifugation tube and spun at 3,000 rpm for 1 min to remove the storage buffer from the column. After placing the column in a new centrifugation tube the DNA sample (about 60 μl) followed by another 40 μl of water were added to the column and the column was spun with 3,000 rpm for 5 min at 4° C. to collect the run through. To concentrate the DNA the eluat from the S300 column was placed on a Microcon 100 membrane (Amicon) and centrifuged until a final volume of 10 μl was achieved. The membrane was washed once with 10 μl of 0.1×TE at 65° C. for 3 min and the fractions were united for use in the next step.

The resulting double-stranded cDNA was in the next step cleaved with a Class IIS restriction enzyme, which was for the purpose of this example Mme I. The reaction was setup by mixing the following components in a final volume of 100 μl:

| | | |
|---|---|---|
| ddcDNA | 50 | μl |
| 10× reaction buffer (NEB) | 10 | μl |
| MmeI (2 U/μl, equal to 3 U/μg DNA) | 1.5 | μl |
| 10× SAM | 2 | μl |
| ddH₂O | to final volume of 100 | μl |

After incubation at 37° C. for 1 h the reaction was terminated by adding 2 μl of 0.5M EDTA, 2 μl of 10% SDS, and 2 μl of 10 μg/μl proteinase K followed by a further incubation at 45° C. for another 15 min. The reaction mixture was then extracted once with the same volume of Tris-equilibrated phenol: chloroform (ratio 1:1) and out of the aqueous phase the DNA was precipitated with isopropanol by adding to 150 μl of the sample 7.5 μl of 5M sodium chloride, 3 μl of 1 μg/μl glycogen, and 150 μl of isopropanol. After incubation at −80° C. for some 30 min, the DNA was collected by centrifugation at 15,000 rpm for 20 min. After having washed the pellet twice with 500 μl 80% ethanol, the DNA was finally re-suspended in 2 μl of 0.1×TE buffer.

After having cleaved the double-stranded cDNA with the Class IIS restriction enzyme MmeI a second linker was ligated to the 2 bp overhang at the cleavage site. This second linker was comprised out of the following two oligonucleotides of 45 bp length and having a XbaI recognition site, which was used in this example for later cloning. However, the invention is not limited to the use of XbaI as other restriction enzymes can be applied for this step with similar efficiency.

Upper-XbaI:

(SEQ ID NO: 6)

Pi-tctagatcaggactcttctatagtgtcacctaaagtctctctctc-
NH₂
Lower-XbaI:

(SEQ ID NO: 7)

gagagagagactttaggtgacactatagaagagtcctgatctagaNN

The two oligonucleotides were obtained from Espec, and purified by acrylamide electrophoresis before being annealed. For annealing a mixture of 2 μg/μl of each oligonucleotide in 100 mM sodium chloride was incubated at 65° C. followed by additional incubations at 45° C. for 5 min, at 37° C. for 10 min, and at 25° C. for 10 min.

The double-stranded linker was then ligated to the cDNA in a reaction mixture containing 2 μl of aforementioned cDNA solution, 4 W of the annealed linker DNA (0.4 μg/μl), and 8 μl of water. Before adding the ligase, the reaction mixture was incubated at 65° C. for 2 min followed by a brief incubation on ice. Then 2 μl of a 10× reaction buffer (NEB), 2 μl of T4 DNA ligase (NEB, 40 U/μl), and 2 μl of water were added, followed by an incubation at 16° C. for 16 h. Heating the reaction mixture to 65° C. for 5 min terminated the ligation reaction.

Ligation products having biotin moistures at the 5′ end were separated from none modified DNA, for which the ligation to the first linker had failed. Streptavidin coated magnetic beads (Dynabeads) were used at this point in a similar way as described before. About 200 μl of the original slurry were incubated under occasional agitation with 5 μg of tRNA in a volume of 200 μl for about 20 min at room temperature. After collection of the beads by a magnetic force, the beads were washed three times with 200 μl of a buffer containing 1M sodium chloride, 0.5 mM EDTA, and 5 mM Tris-HCl pH 7.5, before being re-suspended in 200 μl of the same buffer. After the washing steps the beads were mixed with the aforementioned ligation product, and the resulting slurry was incubated under ongoing agitation at room temperature for 15 min to allow for the binding of the modified DNA to the beads. After the binding reaction was completed, applying a magnetic force collected the beads and the supernatant was removed completely. While being fixed to the bottom of the tube by the magnetic force, the beads were rinsed twice with 200 μl of 1×B&W buffer (10 mM Tris pH 7.5, 1 mM EDTA, 2 M sodium chloride) plus 1×BSA buffer (1 mg/ml provided by NEB), twice with 200 μl of 1×B&W buffer, and finally twice with 200 μl of 0.1×TE.

DNA fragments bound to the magnetic beads by the means of a biotin-streptavidin interaction were released from the beads by treatment with an excess of free biotin. A fresh biotin stock (Sigma) was directly prepared to a final concentration of 1.5% (W/V) in 4 M guanidine thiocyanate, 25 mM sodium citrate, pH 7.0, and 0.5% sodium N-lauroylsarcosinate. The aforementioned beads were re-suspended in 50 μl of the biotin solution and incubated at 45° C. for 30 min under occasional agitation. The supernatant was separated from the beads by applying a magnetic force and collected in a separate tube. The elution step was repeated three times under the same conditions as described above, and all fractions were pooled for the isolation of the cDNA by isopropanal precipitation. For isopropanol precipitation about 250 μl of the sample were mixed with 12.5 μl 5M sodium chloride, 3.5 μl of a 1 μg/μl glycogen solution and 250 μl of isopropanol. After incubation at −80° C. for 30 min the precipitate was collected by centrifugation at 15,000 rpm for 15 min, and the pellet was washed twice with 500 μl of 80% ethanol before being re-suspended in 50 μl 0.1×TE.

The DNA was further purified by gel filtration on a G50 spun column (Amersham Pharmacia Biosciences) according to the maker's directions followed by RNase I and proteinase K treatment To about 100 μl sample derived from the gel filtration 2 μl of RNase I (ProMega) were added, the resulting reaction mixture was incubated for 10 min at 37° C., followed by the addition 2 μl of 10 μg/μl proteinase K, 2 μl of 0.5 M EDTA, and 2 μl of 10% SDS, and an additional incubation of 15 min at 45° C. The reaction mixture was then extracted once with the same volume of Tris-equilibrated phenol:chloroform (ratio 1:1) and out of the aqueous phase the DNA was precipitated with isopropanol by adding to 150 μl of the sample 7.5 μl of 5M sodium chloride, 3 μl of 1 μg/μl glycogen, and 150 μl of isopropanol. After incubation at −80° C. for some 30 min, the DNA was collected by centrifugation at 15,000 rpm for 20 min.

After having washed the pellet twice with 500 μl of 80% ethanol, the DNA was finally re-suspended in 20 μl of 0.1×TE buffer.

Before cloning the DNA fragments were amplified by a PCR step using the following two linker-specific primers, which were obtained from Invitrogen Japan:

| | |
|---|---|
| Primer 1(uni-PCR) | |
| 5′ Bio-gagagagagactttaggtgacacta 3′ | (SEQ ID NO: 8) |
| Primer 2(MmeI-PCR) | |
| 5′ Bio-agagagagacctcgagtaactataa 3′ | (SEQ ID NO: 9) |

The PCR amplification was performed in a total volume of 50 μl and the following setup:

| | | |
|---|---|---|
| DNA Sample | 1 | μl |
| 10× buffer | 5 | μl |
| DMSO | 3 | μl |
| 2.5 mM dNTPs | 12.5 | μl |
| Primer 1(350 ng/μl) | 0.5 | μl |
| Primer 2(350 ng/μl) | 0.5 | μl |
| ddH₂O | 27.5 | μl |
| ExTaq (5 U/μl, TaKaRa) | 0.5 | μl |

After an initial incubation at 94° C. for 1 min, 15 cycles were performed in a thermocycler with 30 sec at 94° C., 1 min at 55° C., 2 min at 70° C. followed by a final incubation 5 min at 70° C. To cover the entire DNA sample 20 PCR reactions were run in parallel to obtain higher yields during the amplification step. The resulting PCR products were then pooled and further purified. To about 600 μl of DNA sample 10 μl of 10 μg/μl proteinase K, 10 μl 0.5 M EDTA, and 10 μl of 10% SDS were added, and incubated for 15 min at 45° C. The reaction mixture was then extracted once with the same volume of Tris-equilibrated phenol:chloroform (ratio 1:1) and out of the aqueous phase the DNA was precipitated with isopropanol by adding to 600 μl of the sample 30 μl of 5M sodium chloride, 3.5 μl of 1 μg/μl glycogen, and 600 μl of isopropanol. After incubation at −80° C. for some 30 min, the DNA was collected by centrifugation at 15,000 rpm for 20 min. After having washed the pellet twice with 500 μl of 80% ethanol, the DNA was finally re-suspended in 50 μl of 0.1×TE buffer.

The PCR products were further purified on a 12% polyacrylamid gel. The appropriate band of 119 bp was visualized by UV and identified by comparison to an appropriate marker and cut out of the gel with a blade, transferred into a tube, crashed by mechanic force, and extracted with 150 μl of a buffer containing 0.5M ammonium acetate, 10 mM magnesium acetate, 1 mM EDTA, pH 8.0, and 0.1% SDS for 1 h at 65° C. The elution step was repeated twice before filtrating the supernatants in a MicroSpin Columns (Amersham Pharmacia Biosciences) by centrifugation at 3,000 rpm in for 2 min. The centrifugation was repeated after applying another 50 μl of 0.1×TE to the column. The resulting extract of about 300 μl was then extracted once with the same volume of Tris-equilibrated phenol:chloroform (ratio 1:1) and out of the aqueous phase the DNA was precipitated with ethanol by adding to 300 μl of the sample 15 μl of 5M sodium chloride, 3.5 μl of 1 μg/μl glycogen, and 750 μl of absolute ethanol. After incubation at −80° C. for some 30 min, the DNA was collected by centrifugation at 15,000 rpm for 20 min. After having washed the pellet twice with 500 μl of 80% ethanol, the DNA was finally re-suspended in 20 μl of 0.1×TE buffer.

Before cloning the DNA fragments were re-amplified by a second PCR step under the same conditions as described above. This second PCR amplification was preferable but not essential to obtain sufficient amounts of DNA for the ligation. Briefly, the PCR amplification was performed in a total volume of 50 μl and the following setup:

| | | |
|---|---|---|
| DNA Sample | 1 | μl |
| 10× buffer | 5 | μl |
| DMSO | 3 | μl |
| 2.5 mM dNTPs | 12.5 | μl |
| Primer 1(350 ng/μl) | 0.5 | μl |
| Primer 2(350 ng/μl) | 0.5 | μl |
| ddH₂O | 27.5 | μl |
| ExTaq (5 U/μl, TaKaRa) | 0.5 | μl |

After an initial incubation at 94° C. for 1 min, 6 cycles were performed in a thermocycler with 30 sec at 94° C., 1 min at 55° C., 2 min at 70° C. followed by a final incubation 5 min at 70° C. To cover the entire DNA sample 20 PCR reactions were run in parallel to obtain higher yields during the amplification step. The resulting PCR products were then pooled and further purified. To about 600 μl of DNA sample 10 μl of 10 μg/μl proteinase K, 10 μl of 0.5 M EDTA, and 10 μl of 10% SDS were added, and incubated for 15 min at 45° C. The reaction mixture was then extracted once with the same volume of Tris-equilibrated phenol:chloroform (ratio 1:1) and out of the aqueous phase the DNA was precipitated with isopropanol by adding to 600 μl of the sample 30 μl of 5M sodium chloride, 3.5 μl of 1 μg/μl glycogen, and 600 μL of isopropanol. After incubation at −80° C. for some 30 min, the DNA was collected by centrifugation at 15,000 rpm for 20 min. After having washed the pellet twice with 500 μl 80% ethanol; the DNA was finally re-suspended in 30 μl of 0.1×TE buffer.

The purified PCR product was for the purpose of this example digested by the restriction enzymes XmaJI and XbaI. Note that cleavage with those two restriction enzymes creates the same overhangs, which can be recombined during the formation of the concatemers. However, the invention is not limited to the use of those two enzymes as other restriction enzymes can be used with similar results. The DNA was first cut with XmaJI in a 100 μl reaction mixture composed of:

| | |
|---|---|
| DNA sample | 30 μl |
| 10× Buffer(Fermantus) | 10 μl |
| XmaJI(10 U/μl, Fermantus) | 10 μl |
| ddH₂O | 50 μl |

After incubation for 1 h at 37° C., 2 μl of 10 μg/μl proteinase K, 2 μl 0.5 M EDTA, and 2 μl 10% SDS were added to the sample, and incubated for 15 min at 45° C. The reaction mixture was then extracted once with the same volume of Tris-equilibrated phenol:chloroform (ratio 1:1) and out of the aqueous phase the DNA was precipitated with isopropanol by adding to 200 μl of the sample 10 μl of 5M sodium chloride, 3.5 μl of 1 μg/μl glycogen, and 200 μl of isopropanol. After incubation at −80° C. for some 30 min, the DNA was collected by centrifugation at 15,000 rpm for 20 min. After having washed the pellet twice with 500 μl 80% ethanol, the DNA was finally re-suspended in 10 μl of 0.1×TE buffer.

For the second digestion with XbaI the aforementioned DNA was then cut with XbaI in a 110 μl reaction mixture composed of:

| | |
|---|---|
| DNA sample | 10 μl |
| 10× Buffer (NEB) | 11 μl |
| 10× BSA (NEB) | 11 μl |
| XbaI(20 Us/μl, NEB) | 11 μl |
| ddH₂O | 67 μl |

After incubation for 1 h at 37° C., 2 μl of 10 μg/μl proteinase K, 2 W 0.5 M EDTA, and 2 μl 10% SDS were added to the sample, and incubated for 15 min at 45° C. The reaction mixture was then extracted once with the same volume of Tris-equilibrated phenol: chloroform (ratio 1:1) and out of the aqueous phase the DNA was precipitated with isopropanol by adding to 200 μl sample 10 μl 5M sodium chloride, 3.5 μl 1 μg/μl glycogen, and 200 μl isopropanol. After incubation at −80° C. for some 30 min, the DNA was collected by centrifugation at 15,000 rpm for 20 min. After having washed the pellet twice with 500 μl 80% ethanol, the DNA was finally re-suspended in 10 μl of 0.1×TE buffer.

The resulting 33 bp DNA fragments were separated from the free DNA ends cut off during the restriction digests by incubation with streptavidin coated magnetic beads, which would retain the biotin-labeled DNA fragments. Streptavidin coated magnetic beads (Dynabeads) were used at this point in a similar way as described before. About 100 μl of the original slurry were incubated under occasional agitation with 5 μg of tRNA for about 20 min at room temperature. After collection of the beads by a magnetic force, the beads were washed three times with 100 μl of 1×B&W. The aforementioned DNA sample was then mixed with the beads, incubated at room temperature for 15 min under ongoing agitation, and the supernatant was taken off after collection of the magnetic beads by magnetic force. The beads were then rinsed one more time with 50 μl 1×B&W buffer, and the collected supernatants were forwarded to isopropanol precipitation of the DNA. To about to 250 μl of sample, 7.5 μl of 5M sodium chloride, 3.5 μl of 1 μg/μl glycogen, and 250 μl of isopropanol were added. After incubation at −80° C. for some 30 min, the DNA was collected by centrifugation at 15,000 rpm for 20 min. After having washed the pellet twice with 500 μl 80% ethanol, the DNA was finally re-suspended in 10 μl of 0.1×TE buffer.

The DNA was further purified by RNase I and proteinase K treatment. To the aforementioned 10 μl sample 5 μl 10×RNase I Buffer (ProMega), 2 μl of RNase I (ProMega), and 33 μl of water were added, the resulting reaction mixture was incubated for 15 min at 37° C., followed by the addition 1 μl of 10 μg/μl proteinase K, 1 μl of 0.5 M EDTA, and 1 μl of 10% SDS, and an additional incubation of 15 min at 45° C. The reaction mixture was then extracted once with the same volume of Tris-equilibrated phenol:chloroform (ratio 1:1) and out of the aqueous phase the DNA was precipitated with isopropanol by adding to 100 μl of the sample 5 μl of 5M sodium chloride, 3.5 μl of 1 μg/μl glycogen, and 100 μl of isopropanol. After incubation at −80° C. for some 30 min, the DNA was collected by centrifugation at 15,000 rpm for 20 min. After having washed the pellet twice with 500 μl of 80% ethanol, the DNA was finally re-suspended in 40 μl of 0.1×TE buffer.

The DNA fragments were further purified on a 12% polyacrylamid gel. The appropriate band of 33 bp as identified by comparing with a suitable molecular weight marker was cut out of the gel with a blade, transferred into a tube, crashed by mechanic force, and extracted with 150 μl of a buffer containing 0.5 M ammonium acetate, 10 mM magnesium acetate, 1 mM EDTA, pH 8.0, and 0.1% SDS for 1 h at 37° C. The extraction step was repeated twice before filtrating the supernatants in a MicroSpin Columns (Amersham Pharmacia Biosciences) by centrifugation at 3,000 rpm in for 2 min. The centrifugation was repeated after applying another 50 μl of 0.1×TE to the column. The resulting extract of about 300 μl was then extracted once with the same volume of Tris-equilibrated phenol: chloroform (ratio 1:1) and out of the aqueous phase the DNA was precipitated with ethanol by adding to 300 μl of the sample 15 μl of 5M sodium chloride, 3.5 μl of 1 μg/μl glycogen, and 750 μl of absolute ethanol. After incubation at −80° C. for some 30 min, the DNA was collected by centrifugation at 15,000 rpm for 20 min. After having washed the pellet twice with 500 μl 80% ethanol, the DNA was finally re-suspended in 4 μl of water.

In the next step of the invention DNA fragments comprising 5′ ends were ligated with each other to form concatemers. For this ligation the following reaction was set up:

| | |
|---|---|
| DNA Sample | 4 μl |
| 10× T4 DNA ligase buffer (New England Biolabs) | 1 μl |
| T4 DNA Ligase (40 U, New England Biolabs) | 1 μl |
| 50% PEG 8000 | 4 μl |

After an incubation of 45 min at 16° C. the reaction was stopped by adding 1 μl 0.5M EDTA, 1 μl 10% SDS, 1 μl 10 μg/μl Proteinase K, and 35 μl of water followed by an additional incubation of 15 min at 45° C. The reaction mixture was then extracted once with the same volume of Tris-equilibrated phenol:chloroform (ratio 1:1) and out of the aqueous phase the DNA was precipitated with isopropanol by adding to 100 μl of the sample 5 μl of 5M sodium chloride, 3.5 μl of 1 μg/μl glycogen, and 100 μl of isopropanol. After incubation at −80° C. for some 30 min, the DNA was collected by centrifugation at 15,000 rpm for 20 min. After having washed the pellet twice with 500 μl of 80% ethanol, the DNA was finally re-suspended in 10 μl of 0.1×TE buffer.

The aforementioned ligation reaction yielded in concatemers of various lengths, and a size selection was performed to clone only concatemers of a suitable length for sequencing, e.g. longer or shorter than 500 bp. Therefore the concatemers were fractionated on an 8% polyacrylamid gel, and bands of a size lager than 500 bp and bands of 200 to 500 bp were cut out of the gel with a blade and further processed separately. After transferring the gel pieces into a tube, those were crashed by mechanic force, and extracted with 150 μl of a buffer containing 0.5M ammonium acetate, 10 mM magnesium acetate, 1 mM EDTA, pH 8.0, and 0.1% SDS for 1 h at 65° C. The extraction step was repeated twice before filtrating the supernatants in a MicroSpin Columns (Amersham Biosciences) by centrifugation at 3,000 rpm in for 2 min. The centrifugation was repeated after applying another 50 μl of 0.1×TE to the column. The resulting extract of about 300 μl was then extracted once with the same volume of Tris-equilibrated phenol:chloroform (ratio 1:1) and out of the aqueous phase the DNA was precipitated with ethanol by adding to 300 μl of the sample 15 μl of 5M sodium chloride, 3.5 μl of 1 μg/μl glycogen, and 750 μl of absolute ethanol. After incubation at −80° C. for some 30 min, the DNA was collected by centrifugation at 15,000 rpm for 20 min.

After having washed the pellet twice with 500 μl 80% ethanol, the DNA was finally re-suspended in 2 μl of water.

In the final cloning step the concatemers were cloned into the vector pZEro-1 (Invitrogen), which was linearized under standard conditions with Xba I and further purified by gel electrophoresis. For this ligation the following reaction was set up:

| | | |
|---|---|---|
| Purified concatemer | 2 | μl |
| XbaI digestion pZErO-1 (100 ng/μl) | 1.25 | μl |
| 10× T4 DNA ligase buffer (New England Biolabs)) | 0.5 | μl |
| T4 DNA Ligase (24 U, New England Biolabs) | 0.6 | μl |
| Water | 0.65 | μl |

After an overnight incubation at 16° C. the reaction was terminated by heat treatment for 5 min at 65° C. followed by adding 1 μl of 0.5M EDTA, 1 μl of 10% SDS, 1 μl of 10 μg/μl Proteinase K, and 30 μl of water followed by an additional incubation of 15 min at 45° C. The reaction mixture was then extracted once with the same volume of Tris-equilibrated phenol:chloroform (ratio 1:1) and out of the aqueous phase the DNA was precipitated with isopropanol by adding to 100 μl of the sample 5 μl of 5M sodium chloride, 3.5 μl of 1 μg/μl glycogen, and 100 μl of isopropanol. After incubation at −80° C. for some 30 min, the DNA was collected by centrifugation at 15,000 rpm for 20 min. After having washed the pellet twice with 500 μl 80% ethanol, the DNA was finally re-suspended in 6 μl of water. Using 1 μl of the aforementioned desalted ligation solution, ElectroMAX™ DH10B™ Cells (Invitrogen) were transformed by electroporation using a Cell-Porator (Biometrer) according to the transformation procedures described in the manufacturer's manual. Transformed bacteria were selected on LB medium containing 50 μg/ml Zeocin (Invitrogen), and positive clones thereof were isolated and further characterized as described in the Examples below.

### Example 2

### Alternative Preparation of 5′ End Specific Tags Involving the Formation of Di-Tags

Preparation of Total RNA from Tissue

In the literature a variety of different approaches for the preparation of RNA have been described, which are known to a person experienced in the state of the art. All such approaches should allow the preparation of a plurality of RNA samples derived from biological materials including tissues and cells, which are suitable for the invention. Below two such procedures are described in detail.

Buffers and Solutions:

a) Solution D: 4M guanidinium thyocyanate, 25 mM sodium citrate (pH7.0), 100 mM 2-mercaptoethanol and 0.5% n-lauryl-sarcosine.

b) RNase-free CTAB/UREA solution: 1% CTAB (Sigma), 4M UREA, 50 mM Tris-HCl (PH 7.0), 1 mM EDTA (pH 8.0).

c) Water equilibrated phenol as described in Molecular Cloning (Sambrook and Russel, 2001).

Phosphate-buffer saline (PBS) as described in Molecular Cloning (Sambrook and Russel, 2001)

5 M Sodium chloride

7 M Guanidium choride

Rnase free dd-water

Protocol for Total RNA Preparation

Dissect the tissue as fast as possible in a cooled dish.

Roughly evaluate the volume of tissue in a 50 ml falcon tube. The best quantity of tissue is between 0.5-1 g of tissue for 20 ml Solution D

Add 2 ml of 2M sodium acetate (pH 4.0) and 16 ml of water-equilibrated phenol.

Mix by a vortex. Add 4 ml of chloroform and shake vigorously by your hands and a vortex.

Let it stay on ice for 15 min.

Centrifuge it at 6,000 rpm for 30 min at 4° C.

Transfer the upper aqueous phase to new tube by pipetting (25 ml) and recover approximately 20 ml thereof.

Precipitate the RNA from the aqueous phase by adding 1 equal volume of Isopropanol (in this case, approximately 20 ml), store on ice for 1 h.

Centrifuge at 7,500 rpm for 15 min at 4° C.: RNA is pelleted by centrifugation.

The pellet is washed twice with 70% ethanol, each time followed by centrifugation at 7,500 rpm for 2 min, in order to remove the SCN salts.

CTAB removal of polysaccharides. Selective CTAB precipitation of mRNA is performed after complete RNA re-suspension in 4 ml of water. Subsequently, 1.3 ml of 5 M NaCl is added and the RNA is then selectively precipitated by adding 16 ml of a CTAB/urea solution.

Centrifuge for 15 min at 7500 rpm (9500×g), discard the aqueous phase.

Resuspend the RNA pellet in 4 ml of 7 M Gunidinum Cloride.

Re-suspended RNA is finally precipitated by adding 8 ml of ethanol. Incubate on −20° C. for 1-2 hours (or longer) and centrifuge for 15 min at 7,500 rpm, 4° C. At the end, wash the pellet with 5 ml of 70% ethanol.

Centrifuge again at 7,500 rpm for 5 min.

Discard the supernatant.

Re-suspend RNA in 500-1000 μl of RNase-free dd-water.

Preparation of a mRNA Fraction from Total RNA

The mRNA fragtion of total RNA preparations can be isolated by the use of commercial kits such as the MACS mRNA isolation kit (Milteny) or polyA-quick (Stratagene), which provide satisfactory yield of mRNA under the recommended conditions. One cycle of oligo-dT selection of the mRNA is sufficient. It is advisable to redissolve the poly-A⁺ RNA at a high concentration of 1 to 2 μg/μl.

Preparation of a Plurality of RNA Samples from a cDNA Library

Alternatively, a plurality of nucleic acids corresponding to the 5′ ends of genes can be obtained from existing cDNA libraries, which were cloned into expression vectors. By standard methods known to a person familiar with the state of the art of molecular biology approaches, from such libraries RNA transcripts can be obtained by in vitro transcription reactions using e.g. a T3, 17 or SP6 RNA polymerase. Such an approach can be performed by first linearization of the plasmid DNA with appropriate restriction endonucleases. The restriction enzyme can be chosen to allow for the transcription of the sense RNA. In the case of libraries obtained in the vector pFLC III (Carninci P, et al., Genomics, 2001 September; 77(1-2):79-90), the vector can be linearized by cleavage with one of the horning endonucleases I-Ceu I or PI-Sce I to avoided a truncation of the inserts. For the digest mix in a tube

| | | |
|---|---|---|
| Plasmid DNA | 100 | μg |
| 10× buffer | 40 | μl |
| Restriction enzyme | 100 | U |
| ddH₂O ad | 400 | μl |

Incubate at appropriate temperature for at least 2 h and analyze 1 μl of the reaction mixture by agarose gel electrophoreses. If the digest is completed, add:

| | |
|---|---|
| 0.5 M EDTA | 8 μl |
| 10% SDS | 8 μl |
| Proteinase K (10 mg/ml) | 5 μl |

Incubate for 15 min at 45° C. before extracting sample with 500 μl phenol/chloroform. The aqueous phase is to be re-extracted twice with 500 μl chloroform. Finally linearized DNA is precipitated with isopropanol or ethanol under standard conditions and dissolved in 50 μl TE.

In Vitro RNA Synthesis:

Mix in a tube under Rnase free conditions:

| | | |
|---|---|---|
| Linearized plasmid DNA | 20 | μg |
| 5× T7 or T3 buffer | 200 | μl |
| 0.1 M DTT | 100 | μl |
| 2 mg/ml BSA | 40 | μl |
| 10 mM rNTPs | 50 | μl |
| T7 or T3 RNA polymerase | 10 | μl |
| ddH₂O ad | 1000 | μl |

Incubate at 37° C. for 3 to 4 h before adding:

| | | |
|---|---|---|
| 10 mM Calcium Chloride | 10 | μl |
| 1 U/μl DNase RQ1 | 5 | μl |

Incubate at 37° C. for 20 min before adding:

| | | |
|---|---|---|
| 0.5 M EDTA | 10 | μl |
| 10 mg/ml Protease K | 5 | μl |

Incubate at 45° C. for 30 min, before addition of Sodium Chlorid to a final concentration of 1M. Phenol/Chloroform extraction followed be re-extraction with Chloroform should be performed under standard conditions, and the RNA transcripts can be finaly collected by Isopropanol or Ethanol precipitation. The pellet is to be resuspended in 200 μl of water or TE. The quality of the RNA transcripts should be confirmed by agarose gel electrophorese and quantification.

First Strand cDNA Synthesis

Buffers and Solutions

Saturated Trehalose, about 80% in water (crystals will remain), low metal content

4.9 M high purity sorbitol

Optionally: Takara GC-Taq buffer

Enzymes and Buffers

RNase H⁻ reverse transcriptase Superscript II (Invitrogen) and buffer or other reverse transcriptases.

Nucleic Acids and Oligonucleotides

Purified, first-strand oligo-dT primer (Sequence for primer used:

(SEQ ID NO: 10)

5′-
GAGAGAGAGAGGATCCTTCTGGAGAGTTTTTTTTTTTTTTTTVN-3′).

Alternatively or additionally, random primer (dN₆-dN₉), where N is any nucleotide mRNA, recommended 2.5 to 25 μg or alternatively, total RNA, 5-50 μg
Radioactive Compounds

[alpha-³²P] dGTP

Protocol A: Trehalose-Sorbitol Enhanced

To prepare the 1^{st} strand cDNA, put together the following reagents in three different 0.5 ml PCR tubes (A, B, and C)

Tube A: in a final volume of 21.3 μl, add the following:

| | | |
|---|---|---|
| mRNA | 2.5-25 | μg |
| or total RNA, | 5-50 | μg |
| 1^{st} strand primer (2 μg/μl) | 14 | μg (7 μl) |
| Total volume: | 22 | μl |

Heat the mixture (mRNA, primer) at 65° C. for 10 min to dissolve the secondary structures of mRNA.

Tube B: in a final volume of 76 μl, add the following:

| | | |
|---|---|---|
| 5× 1^{st} strand buffer | 28.6 | μl |
| 0.1 M DTT | 11 | μl |
| dATP, dTTP, dGTP, and 5-methyl-dCTP 10 mM each | 9.3 | μl |
| 4.9 M sorbitol | 55.4 | μl |
| Saturated trehalose | 23.2 | μl |
| RNase H⁻Superscript II reverse transcriptase (200 U/μl) | 15.0 | μl |
| Final volume: | 142.5 | μl |

Prepare a cycle (on a thermal cycle) with: 40° C., 4 min; 50° C., 2 min; 56° C., 60 min.

If total RNA is used as the starting material, prepare a cycle with:

40° C., 2 min, −0.1° C./sec to 35° C.; 50° C., 2 min; 56° C., 60 min.

Alternatively: prime the cDNA with a random primer (dN₉, N=any nucleotide) at 25° C.

Tube C:

1˜1.5 μl of [alpha-³²P] dGTP.

For a cold-start operate as follows:

Quickly mix tubes A and B on ice.

Transfer in tube C 40 μof the A+B mixture.

Tubes A+B and C should be quickly transferred immediately at 40° C. of the step 1 of the above cycling program to anneal at 40° C. four 4 minutes.

Let the reaction proceed following the thermal cycler setting.

For a hot-start, operate as follows:

Transfer the tubes A, B, C on the thermal cycler

Start the cycling

When the temperature reaches 42° C., quickly mix tubes A and B.

Transfer in tube C 40 μl of the A+B mixture.

Let the reaction proceed following the thermal cycler setting.

Protocol B: GCI-Trehalose-Sorbitol Enhanced

Tube A: in a final volume of 22 μl, add the following:

| | | |
|---|---|---|
| mRNA | 5-25 | μg |
| (precipitate with ethanol and re-suspend directly with the pri- | | |
| mer) or total RNA, up to 50 μg (for the small-scale protocol) | | |
| Purified 1^{st} strand cDNA primer (2 μg/μl)14 μg(7 μl) | | |
| Final volume: | 22 | μl |
| Tube B: add the following: | | |
| 2× GC I (LA Taq) buffer (TaKaRa) | 75 | μl |
| dATP, dTTP, dGTP, and 5-methyl-dCTP, 10 mM each | 4 | μl |
| 4.9 M sorbitol | 20 | μl |
| Saturated trehalose (approximately 80%) | 10 | μl |
| Superscript II reverse transcriptase (200 U/μl) | 15 | μl |
| ddH₂O | 4 | μl |
| Final volume: | 128 | μl |
| Tube C: | | |
| alpha-³²P-dGTP | 1.5 | μl |

For the rest of the procedure, follow exactly the point as in the normal reaction condition. Prepare (in advance) a thermal cycler with the following cycle:

42° C., 30 min; 50° C., 10 min; 55° C., 10 min; 4° C., indefinite time.

Operate as follows:
1) Transfer the tubes A, B, C on the thermal cycler
2) Start the cycling
3) When the temperature reaches 42° C., quickly mix tubes A and B.
4) Transfer in tube C 40 μl of the A+B mixture.
5) Let the reaction proceed following the thermal cycler setting.

At the end, stop the reaction with EDTA at 10 mM final concentration.

Then incorporation of [alpha³ 2 P]GTP is measured and the yield of cDNA is calculated. Calculation of the amount of cDNA by measuring [alpha³² P]GTP is useful for monitoring whether the processes are accurately proceeding or not.

CTAB Precipitation of the First-Strand cDNA

Buffers and Solutions

CTAB solution as described in Example 1

After measuring the radioactivity, transfer both the “hot” and “cold” 1^{st} strand synthesis (tube B and C) to a tube and perform CTAB precipitation as follows.

Mix the tube B and C from the first strand; to the mixture add:

3 μl of 0.5 M EDTA (final concentration of 10 mM)

2 μl of 10 μg/μl Proteinase K

Incubate at 45° C. or 50° C. for at least 15 min, and as long as 1 hour.

To the 128-142 μl volume of the first-strand cDNA reaction, add:

32 μl of 5 M Sodium Chloride (RNase free)

320 μl of CTAB-Urea solution

Incubate at room temperature for 10 min.

Centrifuge at 15,000 rpm for 10 min

Remove supernatant.

Carefully re-suspend with 100 μl of 7M guanidinium chloride

Add 250 μl of ethanol and leave on ice or −20 to −80° C. for 30-60 min

Centrifuge at 15,000 for 10 min. Remove the supernatant.

Subsequently, wash the pellet twice with 800 μl of 80% ethanol. Each time, add 80% ethanol to the tube and centrifuge for 3 min. at 15,000 rpm.

Re-suspend cDNA in water 46 μl.

Cap-Trapping, Oxidation and Biotinylation of the Cap

Buffers and Solutions

1 M sodium acetate buffer, pH 4.5

1M citrate buffer, pH 6.0

NaIO₄, solution >100 mM.

SDS 10%

Biotinylation buffer: 33 mM Sodium citrate, pH 6.0, and 0.33% SDS.
10 mM Biotin Hydrazide long arm (MW=371.51; 3.71 mg/ml=10 mM) in citrate/SDS buffer.

Cap biotinylation: (A) Oxidation of the diol groups of mRNA

In a final volume of 50 to 55 μl, add the following:

The re-suspended cDNA sample

3.3 μl of 1 M sodium acetate buffer, pH 4.5

A freshly prepared solution of NaIO₄ to a final concentration of 10 mM

Incubate on ice in the dark for 45 min.

Finally, precipitate the cDNA:

To simplify the downstream process, add 11 μl of glycerol 80%.

Vortex.

Add 0.5 μl of 10% SDS, 11 μl of 5 M sodium chloride and 61 μl of isopropanol.

Incubate at −20 or −80° C. for 30 min in the dark.

Centrifuge for 15 min at 15,000 rpm.

Remove supernatant.

Add 500 μl of 80% ethanol

Centrifuge at 15,000 rpm for 2-3 min.

Discard the supernatant

Repeat steps 12-13

Re-suspend the cDNA in 50 μl of water.

Biotinylation: (B) Derivatization of the oxidized diol groups

To the cDNA (50 μl), add 160 μl of the dissolved biotin hydrazide long arm in the reaction buffer. Perform the reaction in 210 μl (final volume).

Incubate overnight (10-16 hours) at room temperature (22-26° C.).

Subsequently, to precipitate the biotinylated cDNA, add:

75 μl 1 M Sodium citrate, pH 6.1

5 μl of 5 M Sodium chloride

75 μl of absolute ethanol

Incubate on ice for 1 hour or at −80 or −20° C. for 30 min or longer.

Centrifuge the sample at 15,000 rpm for 10 min

Wash the precipitate twice with 70% or 80% ethanol and centrifuge.

Discard the supernatant and repeat the wash. dissolve the cDNA in 175 μl of TE (1 mM Tris, pH 7.5, 0.1 mM EDTA).

Cap-trapping and releasing the 5′ ends of cDNA enzymes and buffers

RNase ONE (Promega) and its reaction buffer

To the cDNA sample add, in a final volume of 200 μl:

20 μl of RNase I buffer (Promega).

1 units of RNase I (Promega, 5 or 10 U/μl) per each 1 μg of starting mRNA or total RNA (in case of small scale protocol) used for first-strand cDNA synthesis.

Incubate at 37° C. for 30 min.

To stop the reaction, put the sample on ice and add

4 μl 10% SDS and

3 μl of 10 μg/μl Proteinase K.

Incubate at 45° C. for 15 min.

Extract once with 1:1 Tris-equilibrated phenol:chloroform, then load the aqueous phase into Microcon-100.

Perform a back extraction with water and load again into the Microcon-Centricon 100 filter.

Perform one round of Microcon separation

8-b) Dissolve completely the pellet with 20 μl of 0.1×TE

Magnetic Beads Blocking

Materials

Streptavidin-coated MPG (CPG inc., New Jersey)

Buffers and Solutions

Binding buffer: 4.5 M NaCl, 50 mM EDTA, pH 8.0

Special Equipment

A magnetic stand to hold 1.5 ml tubes is required.

To further minimize the non-specific binding of nucleic acids, magnetic beads are pre-incubated with DNA-free tRNA (10 mg/ml).

For each preparation, pre-incubate 500 μl of magnetic beads (per 25 μg of starting mRNA) with 100 μg of tRNA

Incubate on ice for 30 min with occasional mixing.

Separate the beads with a magnetic stand (for 3 min) and remove the supernatant.

Wash for 3 times with 500 μl of binding buffer

5′-Ends cDNA Capture and Release

To capture the full-length cDNA, mix the RNaseI-treated cDNA and wash beads as follows:
1) Re-suspend the beads in 500 μl of wash/binding buffer.
2) Transfer 350 μl of the beads into the tube containing the biotinylated first-strand cDNA
3) After mixing gently rotate the tube for 10 min at 50° C.,
4) Transfer 150 μl of the beads into the tube containing the biotinylated first-strand cDNA and 350 μl of beads.
5) After mixing gently rotate the tube for 20 min at 50° C.

Separate the beads from the supernatant on a magnetic stand.

Washing the Beads

Gently wash the beads with 0.5 ml of the indicated buffer to remove the nonspecifically absorbed cDNAs.

2× with washing/binding solution.

1× with 0.3 M NaCl/1 mM EDTA

2× with 0.4% SDS/0.5 M NaOAc/20 mM Tris-HCl pH 8.5/1 mM EDTA.

2× with 0.5 M NaOAc/10 mM Tris-HCl pH 8.5/1 mM EDTA

Alkali Release (See Below)

Alkali Full-Length cDNA Release from Beads

Add 100 μl of 50 mM NaOH, 5 mM EDTA

Briefly stir and incubate 5 min at RT with occasional mixing.

Separate the magnetic beads and transfer the eluted cDNA on ice.

Repeat the elution cycle with 100 μl of 50 mM NaOH, 5 mM EDTA, two more times until most of the cDNA, 80-90% as measured by monitoring the radioactivity, can be recovered from the beads.

Adding a 5′-End Primable Site to the cDNA

RNase Step

Enzymes and Buffers

RNase ONE™ and its buffer (Promega)

Add 50 μl of 1 M Tris-HCl, pH 7.0 in tubes on ice and mix quickly.

Add 1 μl of RNase I (10 U/μl) and mix quickly.

Incubate at 37° C. for 10 min.

To remove the RNaseI, treat the cDNA with Proteinase K and phenol/chloroform extraction including back extraction.

Add 3 μg of glycogen. Treat the cDNA with one cycle of Microcon-100.

Fractionation of cDNA Before Adding a Primable Site

Materials

Amersham-Pharmacia S-400 spun kit or alternative kits

Buffers and Solutions

Column buffer: 10 mM Tris, pH 8.0, 1 mM EDTA, 0.1% SDS, and 100 mM NaCl

Column buffer without SDS: 10 mM Tris, pH 8.0, 1 mM EDTA and 100 mM NaCl

S400 Spun Column Chromatography

Detailed protocols are described in the kits. This is the running protocol of S400 spun columns.

Shake the column.

Brake the seal and transfer in a 2 ml tube.

Centrifuge at 3,000 rpm 1 min (+4° C.).

Add the cDNA (<20 W volume).

After cDNA, add 80 μl of water.

Centrifuge 2 min at 3000 rpm.

Concentrate by Microcon 100 or precipitate with isopropanol. Recovery should exceed 80%.

SSLLM

Materials

S-300 spun column chromatography kit (Amersham-Pharmacia)

Buffers and Solutions

Column buffer: 10 mM Tris HCl pH 8.0, 1 mM EDTA, 0.1% SDS, 10 mM NaCl.

Enzymes and Buffers

Takara DNA Ligase KIT II.

Nucleic Acids and Oligonucleotides

In the Example given here, the recognition sites for the restriction enzymes Bgl II, Gsu I and Mme I are introduced, however, the invention is not dependent or limited to the use of those restriction enzymes and their recognition sites. In particular, Bgl II (recognition site: AGATCT) can be replaced by any endonuclease suitable for cloning. Other example for such enzyme could include Asc I (recognition site: GGCGCGCC) or Xba I (recognition site: TCTAGA).

Synthesize the following oligonucleotides containing the GsuI restriction site.

Oligonucleotide Bg-Gsu-GN5:

(SEQ ID NO: 11)

5′-Biotin-
AGAGAGAGAACTAGGCTTAATAGGTGACTAGATCTGGAGGNNNNN-3′;
Oligonucleotide Bg-Gsu-N6:

(SEQ ID NO: 12)

5′-Biotin-
AGAGAGAGAACTAGGCTTAATAGGTGACTAGATCTGGAGNNNNNN-3′;
Oligonucleotide Bg-Gsu-down:

(SEQ ID NO: 13)

5′-
P-CTGGAGATCTAGTCACCTATTAAGCCTAGTTCTCTCTCT-NH₂ 3′.

Synthesize the following oligonucleotides containing the Mine I restriction site.

Oligonucleotide Bg-Mme-GN5:

(SEQ ID NO: 14)

5′-Biotin-
AGAGAGAGAACTAGGCTTAATAGGTGACTAGATCTTCCRACGNNNNN-3′;
Oligonucleotide Bg-Mme-N6:

(SEQ ID NO: 15)

5′-Biotin-
AGAGAGAGAACTAGGCTTAATAGGTGACTAGATCTTCCRACNNNNNN-
3′;
Oligonucleotide Bg-Mme-down:

(SEQ ID NO: 16)

5′
P-GTYGGAGATCTAGTCACCTATTAAGCCTAGTTCTCTCTCT-NH₂ 3′.

Where R stands for G or A and Y stands for C or T.

P means that the oligonucleotide must be 5′phosphorylated and NH₂ indicates that an amino-group is added to avoid non-specific ligation and possible hairpin priming.

Oligonucleotides should be purified by acrylamide gel electrophoresis following standard techniques as the first-strand cDNA primer with 10% acrylamide electrophoresis (Sambrook and Russel, 2001). Oligonulceotides should be extracted with phenol/chloroform, chloroform and precipitation with 2 volumes of ethanol as for the first-strand cDNA primer.

Preparation of the linkers.

After OD checking and mixing Bg-Gsu-GN5, Bg-Gsu-N6 and “down” oligonucleotides at ratio 4:1:5, at least 2 μg/μl of DNA; add NaCl at 100 mM final concentration. The oligonulceotides are annealed at 65° C. for 5 min, 45° C. for 5 min, 37° C. for 10 min, 25° C. for 10 min.

Ligation of the First-Strand cDNA

Use 2 μg of linker mixture for up to 1 μg single-strand cDNA Mix linkers and cDNA (final volume: 5 μl)

Heat at 65° C. for 5 min to melt secondary structures of single-strand cDNA

Transfer the linker and cDNA mix on ice.

Add 5 μl of the solution II from the TAKARA DNA ligation Kit.

Add 10 μl of solution I of the kit.

Incubate at 10° C. overnight (at least >10 hours).

At the end of the ligation reaction, stop the reaction by adding 1 μl of 0.5 M EDTA, 1 μl of 10% SDS, 1 μl of 10 mg/ml Proteinase K, 10 μl of water, and incubate at 45° C. for 15 min.

Treat with phenol/chloroform, chloroform and back extract (see appendix) with 60 μl of column buffer

After the ligation, remove the excess linker with S-300 spin column chromatography

1) Shake the column several times and then let it stand upright.

2) Remove the upper cap, then the bottom one.

3) Drain the buffer of the column. Apply 2 ml of the column buffer and drain twice by gravity.

Put the column into a 15 ml centrifuge tube, then centrifuge at 400×g for 2 min in a swing-out rotor at room temperature.

Apply 100 μl of buffer to the column, then centrifuge at 400×g for 2 min. Check the eluted volume. If it is different from the input (100 μl), repeat this step until the eluted volume is the same as the added one.

Set a 1.5 ml tube, after cutting off the cap, into the 15 ml centrifuge tube, and then apply the sample into the column. Centrifuge at 400×g for 2 min.

Collect the eluted fraction in a separate tube. Apply to the column 5001 of buffer, repeat the centrifugation and collect the fraction in a separate tube.

Repeat step 6 for 3 to 5 more times; keep the eluted fractions separate.

Collected fractions should be counted in a scintillation counter. Usually mix the first 2-3 fractions (80% of cpm of cDNA).

Add NaCl to a final concentration of 0.2 M, precipitated the cDNA by adding equivalent of isopropanol.

After precipitation and washing twice with 80% cold ethanol, re-suspend with water.

Second-Strand cDNA

Setting the 2nd strand cDNA program on the thermal cycler as follows:

| | | |
|---|---|---|
| Step 1 | 5 | min at 65° C. |
| Step 2 | 30 | min at 68° C. |
| Step 3 | 72° | C. for 10 min |
| Step 4 | +4° | C. |

Procedure for the Second-Strand-cDNA

Second strand steps, mix in a test tube:

The cDNA

6 μl of LA-Taq polymerase buffer (Takara)

6 μl of 2.5 mM (each) dNTP's (Takara)

0.5 μl of [alpha-³²P] dGTP (optional to follow the incorporation)

After starting the 2nd strand program, put the tube on the thermal cycler.

Add to tube 3 μl of 5 U/μl of LA Polymerase or alternative thermostabe polymerase cocktails, when the samples are at 65° C., during the first step.

Mix quickly but thoroughly

At the end of the cycle of the thermal cycler, stop the reaction by addying 10 mM EDTA (final concentration) and clean up the reaction by Proteinase K treatment, Phenol-chloroform extraction and ethanol precipitation (see Sambrook and Russel, 2001, Molecular Cloning, CSHL press, NY).

Cleavage of cDNA

The cDNA should then be cleaved with the Class IIs restriction enzyme like Gsu I given in this Example.

| | | |
|---|---|---|
| Buffer (10×) (MBI Fermentas) | 10 | μl |
| GsuI(1 U/μl) (use 5 U/μg DNA) | Y | μl |
| ddH₂O | X | μl |
| Final volume | 100 | μl |

Where the Y and X vary depending on the quantity of cDNA

1) Incubate at 37° C. for 1 hour.

2) Added 0.5M EDTA 2 μl.

3) Incubated at 65° C. for 15 min. to inactivate the enzyme

Prepare the Magnetic Beads

Prepare the appropriate quantity of CPG-MPG (Magnetic porous glass beads). The same considerations made for the cap-trapper step are valid at this point

Prepare 200 μl of GPG-beads.

Add 5 μg of tRNA (20 mg/ml).

Incubate at RT for 10-20 min or on ice for 30-60 min, with occasional shaking

Transfer the beads on a magnetic stand for 3 minutes and remove the aqueous phase.

Wash 3 times with: 1M NaCl, 10 mM EDTA use at least a volume equivalent to the starting volume of beads.

Re-suspend beads in 1M NaCl, 10 mM EDTA equivalent to the starting volume of beads.

Release of cDNA Tags

Mixed washed beads and GsuI cut sample.

Incubate at RT for 15 min with occasional gentle mixing

Let it stand on magnetic rack for 3 min.

Recover the supernatant.

Rinse 4× with 500 μl of 1×B&W buffer (binding and washing buffer=5 mM Tris, pH 7.5, 0.5 mM EDTA, and 1 M NaCl) containing 1×BSA (bovine serum albumin) wash.

Wash 2× with 200 μl of 1× ligase buffer (NEB).

Ligating Linkers to Bound cDNA: II Linker Ligation.

In this Example a linker with a recognition site for the restriction enzyme Eco RI is used. However, the invention is not dependent or limited to the use of Eco RI in the second linker. Any other restriction enzyme and its recognition site can be used depending on their convenience for cloning the concatemers.

Oligonucleotides to be Synthesized:

(SEQ ID NO: 17)

5′-
GAGAGAGAGACTTTAGGTGACACTATAGAAGAGTCCTGAGAATTCNN-3′

(SEQ ID NO: 18)

5′-
P-GAATTCTCAGGACTCTTCTATAGTGTCACCTAAAGTCTCTCTCTC-3′

The oligonucleotides are purified and annealed as described for the Linker 1.

LoTE (1 mM Tris, pH 7.5, and 0.1 mM EDTA) 20 μl suspended and add linker II (0.4 μg/μl)

Heat the tube at 65° C. for 5 min, then let sit at room temperature for 15 min.

Add TaKaRa ligation kit II solution II 25 μl and solution 150 μl.

Incubated at 16° C. overnight.

After ligation, wash 4 times with 500 μl 1×B&W buffer containing 1×BSA.

Wash once with 200 μl 1×B&W buffer and twice with 200 μl 1×BglII buffer containing 1×BSA.

Release of cDNA Tags Using the Tagging Enzyme

Add to the sample the following

| | | |
|---|---|---|
| LoTE | X | μl |
| 10× buffer | 10 | μl |
| Bgl II | Y | μl |

Make up the volume to a total of 100 S.

1) Incubate at 37° C. for 1 hour, gently mixing intermittently.

2) Place on magnet, collect supernatant into new tube. The supernatant contains the released 5′ end fragments.

3) Raise volume to 200 μl with LoTE.

To 200 μl of sample (the 5′ ends, tagged with linkers) add:

133 μl 7.5M NH4Oac

3 μl 1 μg/μl glycogen

340 μl Isopropanol

Incubate at −20 or −80° C. for at least 30 min.

Spin for 20 min at 4° C. at 15,000 rpm in a micro-centrifuge. Remove the supernatant. Wash the pellet twice with 80% or 70% ethanol. Centrifuge for 3 min at 15,000 rpm and removed the ethanol wash. At the end, re-suspend in 10 μl LoTE.

Ligating Tags to Form Di-Tags

The 5′ ends of cDNAs are ligated to form di-tags.

1) Add the TaKaRa ligation Kit II solution II 10 μl and solutions 20 μl.

2) Incubate overnight 16° C.

3) Added 10 μl of ddH₂O, 1 μl of 0.5M EDTA, pd of 10% SDS 1 and 1 μl of 10 μg/μl Proteinase K.

4) Incubate at 45° C. for 15 min.

5) Extract once with 1:1 Tris-equilibrated phenol-chloroform aqueous phase. After phenol-chloroform and chloroform, and back extraction.

6) Removal the smallest cDNA fragment with a G-50 spun-column (Size exclusion).

7) precipitate with isopropanol by adding 5 μg of glycogen as carrier.

100 μl sample

67 μl 7.5M NH₄OAc

5 μl glycogen

180 μl Isopropanol

8) Spin for 20 min at 4° C.

9) Wash twice with 80% or 70% ethanol, centrifuge and remove the ethanol.

Cleavage of cDNA with Anchoring Enzyme

1) Re-suspend the sample in 5 μl of LoTE. Add then in order:

| | | |
|---|---|---|
| LoTE | X | μl |
| 10× EcoRI restriction buffer | 5 | μl |
| EcoRI | Y | μl (use 20 Units of EcoRI) |

Bring up the volume to a total of 50 μl.

2) Incubate at 37° C. for 1 hour.

3) Add 1 μl of 0.5M EDTA, 1 μl of 10% SDS 1 and 1 μl of 10 μg/μl Proteinase K 10%.

4) Incubate at 45° C. for 15 min.

5) Extract once with 1:1 Tris-equilibrated phenol:chloroform aqueous phase. After phenol-chloroform and chloroform, and back extraction

6) precipitate with isopropanol by adding 5 μg of glycogen as carrier.

100 μl sample

67 μl 7.5M NH₄OAc

5 μl glycogen

180 μl Isopropanol

8) Spin for 20 min at 4° C.

9) Wash twice with 80% or 70% ethanol, centrifuge and removed the ethanol wash each time.

Ligation of Di-Tags to Form Concatemers

1) Resuspended LoTE 5 mL

2) Added TaKaRa ligation kit II solution II 5 μl and solution II 10 μl.

3) Incubate 1.5 hours at 16° C.

4) Added 0.5M EDTA 1 μl, 10% SDS 1 μl, 10 μg/μl Proteinase K 1 μl.

5) Incubate at 45° C. for 15 min.

6) Extract once with 1:1 Tris-equilibrated phenol:chloroform aqueous phase. After phenol-chloroform and chloroform, and back extraction.

7) precipitate with isopropanol by adding 5 μg of glycogen as carrier.

100 μl sample

67 μl 7.5M NH₄OAc

5 μl glycogen

180 μl Isopropanol

8) Spin for 20 min at 4° C.

9) Wash twice with 80% or 70% ethanol, centrifuge and removed.

Resolved 5 μl ddH₂O.

The above-obtained concatemers are to be further ligated into a cloning vector such as pBlueascript II KS+ (Stratagene). A large variety of cloning vectors are known in the filed, which can be use for invention.

Standard Ligation:

Mix a three time excess of concatemer DNA and 100 ng of an appropriate vector linearized with Eco RI in a volume of 5 μl. Then mix 5 μl of Solution I of DNA Ligation Kit Ver.2 (Takara) to the insert/vector mixture. Incubate the tube at 16° C. for 12-16 h.

Transformation:

To remove salt from the ligation solution, precipitate DNA after the addition of 2 μg of Glycogen (Roche), 20 mM Sodium Chloride and 80% ethanol. The DNA pellet is washed twice with 150 μl of 80% of ethanol, and the pellet is then dissolved in 10 μl of water. Using 1 μl of desalted ligation solution, ElectroMAX™ DH10B™ Cells (Invitrogen) are transformed using Cell-Porator or alike (Biometra) according to the transformation procedures described in the manufacturer's manual. Transformed bacteria are plated on a selective medium and grown overnight. Positive clones are to be isolated from those plates for further characterization of the concatemers.

### Example 3

### Alternative Preparation of 5′ End Specific Tags Involving the Formation of Di-Tags

The invention can be performed with other linkers and restrictions enzymes than specified in the Examples 1 and 2. In one such embodiment, the invention was performed with the following changes, where the same protocols were used as specified in the aforementioned Example 1 if not otherwise noted: RNA samples were prepared as described above and forwarded to first-strand cDNA synthesis. The resulting cDNA-RNA hybrids were fractionated by the Cap-Trapper approach, and cDNA transcript comprising sequences homologous to the 5′ end of mRNA were isolated. Single-stranded cDNA was then ligated to a different first linker comprised of the following oligonucleotides:

Upper Strand:

(SEQ ID NO: 19)

Bio-5′-
agagagagagcttagatgagagtgaCTCGAGCCTAGGtccaacgNNNNN-
3′

(SEQ ID NO: 20)

Bio-5′-
agagagagagcttagatgagagtgaCTCGAGCCTAGGtccaacNNNNNN-
3′
Lower Strand:

(SEQ ID NO: 21)

Pi-5′-
gttggacctaggctcgagtcactctcatctaagctctctctct-H2-3′

The new linker provided recognition sites for the restriction enzymes Xho I (indicated in capital and underlined), Xma JI (indicated in capital), and the tagging enzyme Mme I (indicated in italic).

After the ligation of the linker to the cDNA the second-strand cDNA was prepared, and the double-stranded DNA was cleaved with Mme I to provide 5′ end specific tags. Those tags were then purified on streptavidin-coated magnetic beads (Dynabeads) before addition of the second linker. Again the second linker had a distinct Y-shaped structure compared to the linker used in Examples 1 and 2 as indicated below (SEQ ID NOS: 22 and 23):

```
                                        atcgaaatcccgatctaggctagcg-NH₂
  P-5′-gaattctacgcctctcg
3′-NNcttaagatgcggagagc
                                          gtgaatcgagtttaaggctagcatc-5′
```

This linker was designed to have an Eco RI restriction site (indicated in underlined), and two single-stranded overhangs to allow for strand-specific amplifications. Note that two restriction enzymes with distinct cloning sites were used at this point.

After the ligation of the second linker to the 5′ end tag the resulting DNA fragment comprising the two linkers and one tag was amplified by PCR using the following primers:

| | |
|---|---|
| XM_cDNA_PCR: | |
| 5′-ttagatgagagtgactcgagcctag-3′ | (SEQ ID NO: 24) |
| EcoRI_Y2down_PCR: | |
| 5′-ctacgatcggaatttgagctaagtg-3′ | (SEQ ID NO: 25) |

The PCR product was amplified directly on the streptavidin-coated beads to which the DNA templates were bond to by the means of the biotin-streptavidin interaction. As the PCR primers did not have any biotin moistures, the PCR products could be separated directly from the beads by applying a magnetic force and forwarded to further purification in a 12% polyacrylamid gel.

The purified PCR products were subsequently cleaved by Xma JI, purified in a 12% polyacrylamid gel, and self-ligated to form dimeric tags comprising two 5′ end specific tags and overhangs derived from the second linker at both ends. These dimerization products were further cleaved with Eco RI, and again purified in a 12% polyacrylamid gel before being concatemerized in a ligation reaction. This final gel purification was essential to separate the dimeric tags from the DNA fragments cleaved off during the digestion with Eco RI. The ligation products were fractionated in a 6% polyacrylamid gel, and DNA fragments in the range of 300 to 600 bp and 600 to 4,000 bp were cut out for DNA isolation.

DNA fragments isolated from both fractions were cloned into the Eco RI site of the vector pZero1.0 (Invitrogen), and transformed bacteria were selected on LB medium containing 50 μg/ml Zeocin (Invitrogen). Positive clones thereof were isolated and further characterized as described in the Examples below.

### Example 4

### Sequencing of 5′-End Sequence Tags

After the titer check, bacterial clones were collected by commercially available picking machines (Q-bot and Q-pix; Genetics) and transferred to 384-microwell plates. Transformed E. coli clones holding vector DNA were divided from 384-microwell plates and grown in four 96-deepwell plates. After overnight growth, plasmids were extracted either manually (Itoh M. et al. 1997, Nucleic Acids Res 25:1315-1316) or automatically (Itoh M. et al. 1999, Genome Res. 9:463-470). Sequences were typically run on a RISA sequencing unit (Shimadzu, JAPAN) or a Perlin Elmer-Applied Biosystems ABI 377 in accordance with standard sequencing methodologies such as described by Shibata K. et al. (Genome Res. 2000 10, 1757-71). Sequencing of concatemers was also performed using primers nested in the flanking regions of the cloning vector and a BigDye Terminator Cycle Sequencing Ready Reaction Kit v2.0 (Applied Biosystems) and an ABI3700 (Applied Biosystems) sequencer according to the manufacture's product descriptions. Some concatemers were sequenced from both ends to cover their entire sequence.

Standard primers used for vectors Bluescript and pZero1.0:

| | | |
|---|---|---|
| M13 Reverse | 5′-CAGGAAACAGCTATGAC | (SEQ ID NO: 26) |
| primer: | | |
| M13 (−20) | 5′-GTAAAACGACGGCCAG | (SEQ ID NO: 27) |
| Forward | | |
| primer | | |

### Example 5

### Identification of 5′-End Sequence Tags

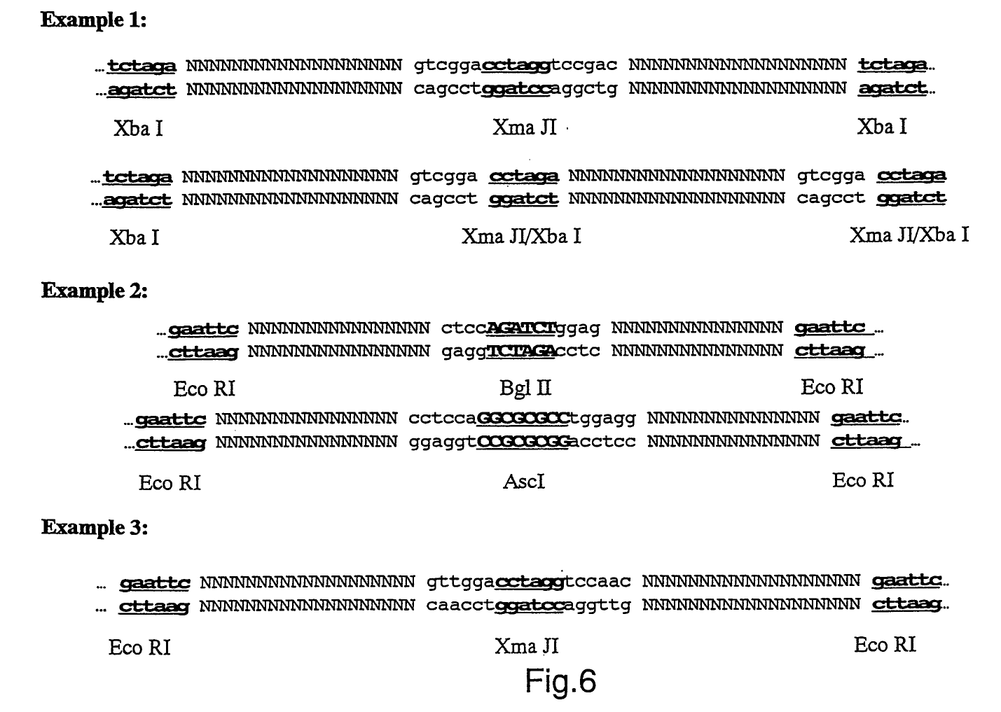

The sequences obtained form concatemers are characterized by the structure of the dimmeric tags and the flanking linker sites as presented in FIG. 6. Defined regions holding the recognition sites for the restriction enzymes used during the cloning steps flank each 5′ end specific sequence tag. Therefore the 5′ end specific sequence tags can be identified by a manual sequence analysis or by an automated process using an appropriate computer program. Individual 5′ end specific sequence tags can be stored in a computer file or a database system.

Initial sequence reads were analyzed by computational means. The individual steps involved in the sequence analysis are described below showing the analysis of one read:

0) Original Sequence:

>zzb21305i03t3.scf 596 0 596 SCF

| | |
|---|---|
| TCGTTAACTATTAGGCGAATTGGGCCCTCTAGGTCGACGAGTTCTCAGCAGAGCC | (SEQ ID NO: 28) |
| GCCGTCTAGAGCCCCGCCCTCCCGGGCCACCGTCGGACCTAGAATAGTTACTCGA | |
| GGTCTCTCGTCGGACCTAGAGTTTTTCGTATGTTTGTCATCGTCGGACCTAGGTCC | |
| GACGGTCCATTCCTGAGAGTCTCTCTAGGTCCGACGAGAGAGAGAGGATCCTTCT | |
| GTCTAGACCCTGACGCCGGAACCGCACCGTCGGACCTAGGTCCGACGGAAAAGC | |
| AGCTTCCTCCACTCTAGGTCCGACGGTGTGTGTGTGTGTGCGTGTTCTAGAGACT | |
| GGTTCAGATCAAAAGTCGTCGGACCTAGGTCCGACGGGGCTGGTGAGATGGCTC | |
| AGTCTAGATGCATGCTCGAGCGGCCGCCAGTGTGATGGATATCTGCCNAATNCC | |
| AGCACACCGGCGCGCGCNACCAGTGGATCCGAGCCCGGTACCAAGCTTGATGCA | |
| TACCTCGAGTATCCTATACTGTCACCTAAATAGCTTGGGGTAATCATGGTCATAG | |
| CTGTCTCCTGTGTGAAATTGTTATCCGCTCAAAATTCCCAACAACATAG | |

1) pZErO-1 vector portions of sequences were masked using program called “cross_match”. X stands for “masked”.

>zzb21305i03t3.scf 596 0 596 SCF

TCGTTAXXXXXXXXXXXXXXXXXXXXXXXXXXGTCGACGAGTTCTCAGCAGAG
CCGCCGTCTAGAGCCCCGCCCTCCCGGGCCACCGTCGGACCTAGAATAGTTACTC
GAGGTCTCTCGTCGGACCTAGAGTTTTTCGTATGTTTGTCATCGTCGGACCTAGG
TCCGACGGTCCATTCCTGAGAGTCTCTCTAGGTCCGACGAGAGAGAGAGGATCC
TTCTGTCTAGACCCTGACGCCGGAACCGCACCGTCGGACCTAGGTCCGACGGAA
AAGCAGCTTCCTCCACTCTAGGTCCGACGGTGTGTGTGTGTGTGCGTGTTCTAGA
GACTGGTTCAGATCAAAAGTCGTCGGACCTAGGTCCGACGGGGCTGGTGAGATG
GCTCAGXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXX
XXXXXXXXXXG

2) Look for linker sequences using “cross_match”

| | |
|---|---|
| Linker sequence | |
| according to Example 1: | |
| “NCTAGGTCCGAC” | (SEQ ID NO: 29) |
| Linker sequence | |
| according to Example 3: | |
| “NGTTGGACCTAGGTCCAACN” | (SEQ ID NO: 30) |

Linkers found using “cross_match” (excerpts from output):

| | |
|---|---|
| linker1 | |
| TCTAGGTCCGACG 86-98 13-1 C | (SEQ ID NO: 31) |
| linker2 | |
| TCTAGGTCCGACG 118-130 13-1 C | |
| linker3 | |
| CCTAGGTCCGACG 151-163 13-1 C | (SEQ ID NO: 32) |
| linker4 | |
| CCTAGGTCCGACG 158-170 1-13 | |
| linker5 | |
| TCTAGGTCCGACG 190-202 1-13 | |
| linker6 | |
| CCTAGGTCCGACG 249-261 13-1 C | |
| linker7 | |
| CCTAGGTCCGACG 256-268 1-13 | |
| linker8 | |
| TCTAGGTCCGACG 288-300 1-13 | |
| linker9 | |
| CCTAGGTCCGACG 347-359 13-1 C | |
| linker10 | |
| CCTAGGTCCGACG 354-366 1-13 | |

3) Using output from “cross_match”. Tag extraction program identifies location and direction of linkers in sequences.

−−−−−−−−−−−−− means linker in reverse direction

+++++++++++++ means linker in positive direction

−−−−−−−−−−++++++++++ dimeric linker (reverse and forward direction)

>zzb21305i03t3 596

TCGTTAXXXXXXXXXXXXXXXXXXXXXXXXXXGTCGACGAGTTCTCAGCA
GAGCCGCCGTCTAGAGCCCCGCCCTCCCGGGCCAC−−−−−−−−−−−−−AT
AGTTACTCGAGGTCTCT−−−−−−−−−−−−−GTTTTTCGTATGTTTGTCAT
−−−−−−−−−−++++++++++GTCCATTCCTGAGAGTCTC+++++++++++
++AGAGAGAGAGGATCCTTCTGTCTAGACCCTGACGCCGGAACCGCAC−
−−−−−−−−++++++++++GAAAGCAGCTTCCTCCACA+++++++++++++
GTGTGTGTGTGTGTGCGTGTTCTAGAGACTGGTTCAGATCAAAAGT−−−−
−−−−−−++++++++++GGGCTGGTGAGATGGCTCAGXXXXXXXXXXXXXX
XXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXG

4) Script looked for restriction enzyme site at possible locations. For example, a gap between two linkers (or linker-vector) that are long enough for two tags.

“TCTAGA” for monomer

“GAATTC” for dimer

It was masked with “******”

>zzb21305i03t3 596

TCGTTAXXXXXXXXXXXXXXXXXXXXXXXXXXGTCGACGAGTTCTCAGCA
GAGCCGCCG******GCCCCGCCCTCCCGGGCCAC−−−−−−−−−−−−−AT
AGTTACTCGAGGTCTCT−−−−−−−−−−−−−GTTTTTCGTATGTTTGTCAT
−−−−−−−−−−++++++++++GTCCATTCCTGAGAGTCTC+++++++++++
++AGAGAGAGAGGATCCTTCTG******CCTGACGCCGGAACCGCAC−−
−−−−−−−−GAAAAGCAGCTTCCTCCAC+++++++++++++
GTGTGTGTGTGTGTGCGTGT******GACTGGTTCAGATCAAAAGT−−−−
−−−−−−++++++++++GGGCTGGTGAGATGGCTCAGXXXXXXXXXXXXXX
XXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXG

5) Script extracted tags from the sequences that were not masked from vector, linker, restriction enzyme site. Tags also must be a) at right size (19-20 bp) and b) located right next to linker with right direction (+++++++ tag or tag −−−−−−−−−)

| | | |
|---|---|---|
| tag1 20 | GTGGCCCGGGAGGGCGGGGC | (SEQ ID NO: 33) |
| tag2 19 | AGAGACCTCGAGTAACTAT | (SEQ ID NO: 34) |
| tag3 20 | ATGACAAACATACGAAAAAC | (SEQ ID NO: 35) |
| tag4 19 | GTCCATTCCTGAGAGTCTC | (SEQ ID NO: 36) |
| tag5 20 | AGAGAGAGAGGATCCTTCTG | (SEQ ID NO: 37) |
| tag6 20 | GTGCGGTTCCGGCGTCAGGG | (SEQ ID NO: 38) |
| tag7 19 | GAAAAGCAGCTTCCTCCAC | (SEQ ID NO: 39) |
| tag8 20 | GTGTGTGTGTGTGTGCGTGT | (SEQ ID NO: 40) |
| tag9 20 | ACTTTTGATCTGAACCAGTC | (SEQ ID NO: 41) |
| tag10 20 | GGGCTGGTGAGATGGCTCAG | (SEQ ID NO: 42) |

The following definitions were used to categorize the tags:

“Good tag” meant:

1) Not a vector sequence (Step 1)

2) Not a linker sequence (Step 2)

3) Not a restriction site (Step 4)

4) Next to linker with correct direction (Step 5)

5) At right sizes (19-20 bp). (Step 5)

In future, quality value will play a role too.

Program outputs linker information, masked sequences, tag sequences.
-“junk” meant:

When program/script could not recognize restriction enzyme site or linker sequences (because of bad quality value), sequences will be considered as junk. Also vector sequences that were not masked properly (because of bad quality value) were considered as junk too.

Below the output of a computer based analysis of a sequencing read is given. The sequence read was obtained from a clones prepared according to the protocol given in Example 1. Note that XmaJI and Xba I create the same overhang after digestion, and therefore in this example sequence many linker sites are derived from recombined XmaJI/XbaI sides. The program identified linker sites as indicated by symbols and highlighted the 5′ end specific sequence tags as described above. Note in the list for the 5′ end specific tags given below, the program automatically remove the first base as this position is primed for artifacts due to the template free site activity of the reverse transcriptase.

>zzb2 106i09t3.scf 569 (monomer)

CATTAGGGGATTGGGCCC+++++++++++++GTACCTCCTCGCATCCCGC
******ACCTTCGACACGCACACCAC−−−−−−−−−−++++++++++ATGG
ACCGAGGGCCCCAGCC+++++++++++++CGGATCGGGTGGGTCGGAC**
****ACGAACTGCTGCGACCTCT−−−−−−−−−−−−−CACAGCGCCGGCTC
CGGAGA−−−−−−−−−−−−−CTCGGAGCCTGCAAAGTCT−−−−−−−−−−−−
−TCCGGCGCTGCGGCAGCTCC−−−−−−−−−−−−−GCGACCAGGTCCGACG
GTGT−−−−−−−−−−−−−GACTCTGGGCGAGAACGTCT−−−−−−−−−−+++
+++++++GCCGTTCCTTGCTTGCTGGA******CTGAGCTAAATCCCCAA
CCC−−−−−−−−−−++++++++++GAGTAACTATAACGGTCCT******GC
GAGCTCCAGGCGGAATC−−−−−−−−−−−−−ACCCGGGGGGCGGGACTAAC
CGTCGGAC+++++++++++++AGGGACCGCTGCGGTCCGXXXXXXXXXXX
GAGCTCCAGGCGGAATC−−−−−−−−−−−−−ACCCGGGGGGCGGGACTAAC
CGTCGGAC+++++++++++++AGGGACCGCTGCGGTCCGXXXXXXXXXXX
XXXXXXXXXXXXXXXXXXN

linker1 19 31

linker2 77 89 C

linker3 84 96

linker4 117 129

linker5 174 186 C

linker6 207 219 C

linker7 239 251 C

linker8 272 284 C

linker9 305 317 C

linker10 338 350 C

linker11 345 357

linker12 404 416 C

linker13 411 423

linker14 468 480 C

linker15 509 521

| | |
|---|---|
| tag1 F 19 | |
| GTACCTCCTCGCATCCCGC | (SEQ ID NO: 43) |
| tag2 R 20 | |
| GTGGTGTGCGTGTCGAAGGT | (SEQ ID NO: 44) |
| tag3 F 20 | |
| ATGGACCGAGGGCCCCAGCC | (SEQ ID NO: 45) |
| tag4 F 19 | |
| CGGATCGGGTGGGTCGGAC | (SEQ ID NO: 46) |
| tag5 R 19 | |
| AGAGGTCGCAGCAGTTCGT | (SEQ ID NO: 47) |
| tag6 R 20 | |
| TCTCCGGAGCCGGCGCTGTG | (SEQ ID NO: 48) |
| tag7 R 19 | |
| AGACTTTGCAGGCTCCGAG | (SEQ ID NO: 49) |
| tag8 R 20 | |
| GGAGCTGCCGCAGCGCCGGA | (SEQ ID NO: 50) |
| tag9 R 20 | |
| ACACCGTCGGACCTGGTCGC | (SEQ ID NO: 51) |
| tag10 R 20 | |
| AGACGTTCTCGCCCAGAGTC | (SEQ ID NO: 52) |
| tag11 F 20 | |
| GCCGTTCCTTGCTTGCTGGA | (SEQ ID NO: 53) |
| tag12 R 20 | |
| GGGTTGGGGATTTAGCTCAG | (SEQ ID NO: 54) |
| tag13 F 19 | |
| GAGTAACTATAACGGTCCT | (SEQ ID NO: 55) |
| tag14 R 19 | |
| GATTCCGCCTGGAGCTCGC | (SEQ ID NO: 56) |
| tag15 F 18 | |
| AGGGACCGCTGCGGTCCG | (SEQ ID NO: 57) |
| zzb21106i09t3 junk 18 | |
| CATTAGGGGATTGGGCCC | (SEQ ID NO: 58) |
| zzb21106i09t3 junk 28 | |
| ACCCGGGGGGCGGGACTAACCGTCGGAC | (SEQ ID NO: 59) |
| zzb21106i09t3 junk 1 N | |

Similar to the example shown above, the sequence example given below was derived from a concatemer prepared according to Example 3, and analyse by the means of the same software solution as described above.

>zzc20401c11t3 607 (dimer)

TGATAAGGCAATGGCCTCTAATGCTGXXXXXXXXXXXXXXXXXXXXXXXX
XXXXXXXXXXXXGCCGCCGCGCCTTCCGCGTC−−−−−−−−−−++++++++
++GAGGGCCGCCGCCCGCCCTCC******AGTTTTTTTTTTTTTTTTG−−
−−−−−−−−++++++++++GGGCAGAGCGAGCAGAGCCT******GTCTGT
CAGAATCAGAAGT−−−−−−−−−−++++++++++GCTTTGCAGACGCCACT
GT******AAAGTCCACCTGGACTTTCC−−−−−−−−−−++++++++++CC
TGCGCGGCCTCGGCGGC******AACTCTGTTATACACTAAC−−−−−−−−
−−++++++++++AGAGACTGAACAGCGGGCGA******CAGCCATCTTGC
CCCACCT−−−−−−−−−−++++++++++GCTTGCCTTCTGGCCATGCC***
***CCCCCCTCTATGCGTGCGTC−−−−−−−−−−++++++++++AGTGTGG
CTGTTCCATGGNXXXXXXXXXXXXXXXXXXXXXXXXXXXXXXXXXXXXXX
XXXXXXXXXXXXXXXXXXXXXXXXXXXXXXXXXXXXXXXXXXXXXXXXXX
XXXXXXG

linker1 83-102 1-20

linker2 149-168 1-20

linker3 214-233 1-20

linker4 279-298 1-20

linker5 343-362 1-20

linker6 408-427 20-1 C

linker7 474493 20-1 C

| | | |
|---|---|---|
| tag1 20 | GACGCGGAAGGCGCGGCGGC | (SEQ ID NO: 60) |
| tag2 21 | GGAGGGCGGGCGGCGGCCCTC | (SEQ ID NO: 61) |
| tag3 19 | CAAAAAAAAAAAAAAAACT | (SEQ ID NO: 62) |
| tag4 20 | AGGCTCTGCTCGCTCTGCCC | (SEQ ID NO: 63) |
| tag5 19 | ACTTCTGATTCTGACAGAC | (SEQ ID NO: 64) |
| tag6 19 | ACAGTGGCGTCTGCAAAGC | (SEQ ID NO: 65) |
| tag7 20 | GGAAAGTCCAGGTGGACTTT | (SEQ ID NO: 66) |
| tag8 19 | GCCGCCGAGGCCGCGCAGG | (SEQ ID NO: 67) |
| tag9 19 | GTTAGTGTATAACAGAGTT | (SEQ ID NO: 68) |
| tag10 20 | TCGCCCGCTGTTCAGTCTCT | (SEQ ID NO: 69) |
| tag11 19 | AGGTGGGGCAAGATGGCTG | (SEQ ID NO: 70) |
| tag12 20 | GGCATGGCCAGAAGGCAAGC | (SEQ ID NO: 71) |
| tag13 20 | GACGCACGCATAGAGGGGGG | (SEQ ID NO: 72) |
| tag14 19 | NCCATGGAACAGCCACACT | (SEQ ID NO: 73) |
| junk1 26 | TGATAAGGCAATGGCCTCTAATGCTG | (SEQ ID NO: 74) |
| junk2 1 | G | |

Note that in both example sequence reads the length of the 5′ end specific tags varies in length, because Mme I cut with some frequency shorter DNA fragments. A statistical analysis of 5′ end specific tags showed that in the examples about 45% of the tags had a length of 21 bp and additional 44% of the tags had a length of 20 bp. Also for the use of the Class IIS enzyme GsuI some variations in the sequence length have been seen, though about 92% of the cases 16 bp DNA fragments were obtained.

### Example 6

### Characterization of 5′-End Sequence Tags

5′ end specific sequence tags can be analyzed for their identity by standard software solutions to perform sequence alignments like NCBI BLAST (http://wvmw.ncbi.nlm.nih.gov/BLAST/), FASTA, available in the Genetics Computer Group (GCG) package from Accelrys Inc. (http://www.accelrys.com/) or alike. Such software solutions allow for an alignment of 5′ end specific sequence tags among one another to identify unique or non-redundant tags, which can be further used in Database searches and building a 5′-end sequence database.

Gene Identification Using a 5′-End Sequence Database

An example of a BLAST search in GenBank using a 5′ end specific tag is given below: The 16 bp tag (5′-ACC TCC CTC CGC GGA G) (SEQ ID NO: 75) is derived from the 5′ end of Human TGF-b1: JBC 264 (1989) 402-408.

Query=(16 letters)(ACCTCCCTCCGCGGAG)

Database: All GenBank+EMBL+DDBJ+PDB sequences (but no EST, STS, GSS, or phase 0, 1 or 2 HTGS sequences)
1,205,903 sequences; 5,297,768,116 total letters
   Score E
   Sequences Producing Significant Alignments: (Bits) Value

gi|0863872|ref|NM_{—}000660.1| Homo sapiens transforming grow . . . 32 1.1

gi|18590091|ref|XM_{—}4085882.1| Homo sapiens similar to transf . . . 32 1.1

gi|11424057|ref|XM_{—}4008912.11 Homo sapiens transforming grow . . . 32 1.1

gi|7684381|gb|AC011462.4|AC011462 Homo sapiens chromosome 1 . . . 32 1.1

gi|15027087|emb|AL89894.4|LMFLCHR4 A Leishmania major Fried . . . 32 1.1

gi|1943914|gb|U70540.1|LMU70540 Leishmania mexicana amazone . . . 32 1.1

gi|37097|emb|X05839.1|HSTGFBG1 Human transforming growth fa. 32 1.1

gi|37092|emb|X02812.1|HSTGFB1 Human mRNA for transforming g . . . 32 1.1

gi|340526|gb|J04431.1|HUMTGFB1PR Homo sapiens transforming . . . 32 1.1

Alignments

>gi|10863872|ref|NM_{—}000660.1| Homo sapiens transforming growth factor, beta 1 (Camurati-Engelmann disease) (TGFB1), mRNA
Length=2745

Score=32.2 bits (16), Expect=1.1

Identities=16/16 (100%)

Strand=Plus/Plus

| | |
|---|---|
| Query: 1 | acctccctccgcggag 16 |
| | \|\|\|\|\|\|\|\|\|\|\|\|\|\|\|\| |
| Sbjct: 1 | acctccctccgcggag 16 |

>gi|18590091|ref|XM_{—}085882.1| Homo sapiens similar to transforming growth factor, beta 1 (H. sapiens) (LOC147760), mRNA
Length=697

Score=32.2 bits (16), Expect=1.1

Identities=16/16 (100%)

Strand=Plus/Plus

| | |
|---|---|
| Query: 1 | acctccctccgcggag 16 |
| | \|\|\|\|\|\|\|\|\|\|\|\|\|\|\|\| |
| Sbjct: 7 | acctccctccgcggag 22 |

>gi|11424057|ref|XM_{—}008912.1| Homo sapiens transforming growth factor, beta 1 (TGFB1), mRNA
Length=2741

Score=32.2 bits (16), Expect 1.1

Identities=16/16 (100%)

Strand=Plus/Plus

| | |
|---|---|
| Query: 1 | acctccctccgcggag 16 |
| | \|\|\|\|\|\|\|\|\|\|\|\|\|\|\|\| |
| Sbjct: 1 | acctccctccgcggag 16 |

Database: All GenBank+EMBL+DDBJ+PDB sequences (but no EST, STS, GSS, or phase 0, 1 or 2 HTGS sequences)

Posted date: Apr. 9, 2002 10:59 AM

Number of letters in database: 1,002,800,820

Number of sequences in database: 1,205,903

| Lambda | K | H |
|---|---|---|
| 1.37 | 0.711 | 1.31 |
| Gapped | | |
| 1.37 | 0.711 | 1.31 |

Matrix: blastn matrix: 1-3

Gap Penalties: Existence: 5, Extension: 2

Number of Hits to DB: 6901

Number of Sequences: 1205903

Number of extensions: 6901

Number of successful extensions: 1479

Number of sequences better than 10.0: 16

length of query: 16

length of database: 5,297,768,116

effective HSP length: 15

effective length of query: 1

effective length of database: 5,279,679,571

effective search space: 5279679571

effective search space used: 5279679571

T: 0

A: 30

X1: 6 (11.9 bits)

X2: 15 (29.7 bits)

S1: 12 (24.3 bits)

S2: 15 (30.2 bits)

Top of Form

| | |
|---|---|
| 1: NM_000660. Homo sapiens | Related Sequences, OMIM, |
| | Protein, Pub [symbol] |
| tran . . . [gi: 10863872] | Taxonomy, UniSTS, LinkOut |

LOCUS NM_{—}000660 2745 bp mRNA linear PRI 13-Feb.-2002

DEFINITION Homo sapiens transforming growth factor, beta 1 (Camurati-Engelmann disease) (TGFB1), mRNA.

ACCESSION NM_{—}000660

VERSION NM_{—}000660.1 GI:10863872

KEYWORDS

SOURCE human.

ORGANISM Homo sapiens
Eukaryota; Metazoa; Chordata; Craniata; Vertebrata; Euteleostomi; Mammalia; Eutheria; Primates; Catarrhini; Hominidae; Homo.
   Reference 1 (Bases 1 to 2745)
AUTHORS Derynck, R., Jarrett, J. A., Chen, E. Y., Eaton, D. H., Bell, J. R., Assoian, R. K., Roberts, A. B., Sporn, M. B. and Goeddel, D. V.
TITLE Human transforming growth factor-beta complementary DNA sequence and expression in normal and transformed cells
JOURNAL Nature 316 (6030), 701-705 (1985)
MEDLINE 85296301
   Reference 2 (Bases 1 to 2745)
AUTHORS Sporn, M. B., Roberts A. B., Wakefield, L. M. and Assoian, R. K.
TITLE Transforming growth factor-beta: biological function and chemical structure
JOURNAL Science 233 (4763), 532-534 (1986)
MEDLINE 86261803
PUBMED 3487831
   Reference 3 (Bases 1 to 2745)
AUTHORS Chang, N. S., Mattison, J., Cao, H., Pratt, N., Zhao, Y. and Lee, C.
TITLE Cloning and characterization of a novel transforming growth factor-beta1-induced TIAF1 protein that inhibits tumor necrosis factor cytotoxicity
JOURNAL Biochem. Biophys. Res. Commun. 253 (3), 743-749 (1998)
MEDLINE 99119079
PUBMED 9918798
   Reference 4 (Bases 1 to 2745)
AUTHORS Ghadami, M., Makita, Y., Yoshida, K., Nishimura, G., Fukushima, Y., Wakui, K., Ikegawa, S., Yamada, K., Kondo, S., Niikawa, N. and Tomita, H.
TITLE Genetic mapping of the Camurati-Engelmann disease locus to chromosome 19q13.1-q13.3
JOURNAL Am. J. Hum. Genet. 66 (1), 143-147 (2000)
MEDLINE 20100617
PUBMED 10631145
   Reference 5 (Bases 1 to 2745)
AUTHORS Vaughn, S. P., Broussard, S., Hall, C. R., Scott A., Blanton, S. H., Milunsky, J. M. and Hecht, J. T.
TITLE Confirmation of the mapping of the Camurati-Englemann locus to 19q13.2 and refinement to a 3.2-cM region
JOURNAL Genomics 66 (1), 119-121 (2000)
MEDLINE 20304762
PUBMED 10843814
   Reference 6 (Bases 1 to 2745)
AUTHORS Lim, J. M., Kim, J. A., Lee, J. H. and Joo, C. K.
TITLE Downregulated expression of integrin alpha6 by transforming growth factor-beta(1) on lens epithelial cells in vitro
JOURNAL Biochem. Biophys. Res. Commun. 284 (1), 3341 (2001)
MEDLINE 21268957
PUBMED 11374867

COMMENT PROVISIONAL REPSEO: This record has not yet been subject to final NCBI review. The reference sequence was derived from X02812.1.

| FEATURES | Location/Qualifiers |
|---|---|
| source | 1 . . . 2745 |
| | /organism=“Homo sapiens” |
| | /db_xref=“taxon:9606” |
| | /chromosome=“19” |
| | /map=“19q13.1” |
| gene | 1 . . . 2745 |
| | /gene=“TGFB1” |
| | /note=“TGFB; DPD1; CED” |
| | /db_xref=“LocusID:7040” |
| | /db_xref=“MIM:190180” |
| misc_feature | 37 . . . 113 |
| | /note=“pot.hairpin loops-forming region” |
| variation | 72 |
| | /allele=“-” |
| | /allele=“C” |
| | /db_xref=“dbSNP:1800999” |
| variation | 79 |
| | /allele=“-” |
| | /allele=“C” |
| | /db_xref=“dbSNP:1799753” |
| CDS | 842 . . . 2017 |
| | /gene=“TGFB1” |
| | /note=“transforming growth factor, beta 1; diaphyseal |
| | dysplasia 1, progressive (Camurati-Engelmann disease)” |
| | /codon_start=1 |
| | /db_xref=“LocusID:7040” |
| | /db_xref=“MIM:190180” |
| | /product=“transforming growth factor, beta 1 |
| | (Camurati-Engelmann disease)” |
| | /protein_id=“NP_000651.1” |
| | /db_xref=“GI:10863873” |

| | |
|---|---|
| /translation = “MPPSGLRLLPLLLPLLWLLVLTPGPPAAGLSTCKTIDMELVKRKRIEAIR | (SEQ ID NO: 77) |
| GQILSKLRLASPPSQGEVPPGPLPEAVLALYNSTRDRVAGESAEPEPEPEADYYAKEV | |
| TRVLMVETHNEIYDKFKQSTHSIYMFFNTSELREAVPEPVLLSRAELRLLRRLKLKVE | |
| QHVELYQKYSNNSWRYLSNRLLAPSDSPEWLSFDVTGVVRQWLSRGGEIEGFRLSA | |
| HCSCDSRDNTLQVDINGFTTGRRGDLATIHGMNRPFLLLMATPLERAQHLQSSRHRR | |
| ALDTNYCFSSTEKNCCVRQLYIDFRKDLGWKWIHEPKGYHANFCLGPCPYIWSLDT | |
| QYSKVLALYNQHNPGASAAPCCVPQALEPLPIVYYVGRKPKVEQLSNMIVRSCKCS” | |

| misc_feature | 863 . . . 910 | | | |
|---|---|---|---|---|
| | /note=“pot. core sequence of signal peptide | | | |
| | (aa −272 to −257)” | | | |
| variation | 870 | | | |
| | /allele=“C” | | | |
| | /allele=“T” | | | |
| | /db_xref=“dbSNP:1982073” | | | |
| variation | 915 | | | |
| | /allele=“C” | | | |
| | /allele=“G” | | | |
| | /db_xref=“dbSNP:1800471” | | | |
| misc_feature | 938 . . . 1600 | | | |
| | /note=“TGFb_propeptide; Region: TGF-beta | | | |
| | propeptide” | | | |
| misc_feature | 953 | | | |
| | /note=“pot. altern. translation start site” | | | |
| misc_feature | 1035 . . . 1043 | | | |
| | /note=“put. glycosylation site” | | | |
| misc_feature | 1247 . . . 1255 | | | |
| | /note=“put. glycosylation site” | | | |
| misc_feature | 1370 . . . 1378 | | | |
| | /note=“put. glycosylation site” | | | |
| variation | 1632 | | | |
| | /allele=“C” | | | |
| | /allele=“T” | | | |
| | /db_xref=“dbSNP:1800472” | | | |
| mat_peptide | 1679 . . . 2014 | | | |
| | /product=“mature TGF-beta (aa 1-112)” | | | |
| misc_feature | 1715 . . . 2014 | | | |
| | /note=“TGF-beta; Region: Transforming growth | | | |
| | factor beta like domain” | | | |
| misc_feature | 1721 . . . 2014 | | | |
| | /note=“TGFB; Region: Transforming growth | | | |
| | factor-beta (TGF-beta) family” | | | |
| misc_feature | 2018 . . . 2096 | | | |
| | /note=“GC-rich region” | | | |
| promoter | 2097 . . . 2103 | | | |
| | /note=“TATA-box-like region” | | | |
| misc_feature | 2517 . . . 2522 | | | |
| | /note=“put. polyadenylation signal” | | | |
| polyA_site | 2539 | | | |
| | /note=“polyadenylation site” | | | |
| BASE COUNY | 527 a | 938 c | 801 g | 479 t |

ORIGIN

(SEQ ID NO: 76)

| | |
|---|---|
| 1 | acctccctcc gcggagcagc cagacagcga gggccccggc |
| | cgggggcagg ggggacgccc |
| 61 | cgtccggggc accccccccg gctctgagcc gcccgcgggg |
| | ccggcctcgg cccggagcgg |
| 121 | aggaaggagt cgccgaggag cagcctgagg ccccagagtc |
| | tgagacgagc cgccgccgcc |
| 181 | cccgccactg cggggaggag ggggaggagg agcgggagga |
| | gggacgagct ggtcgggaga |
| 241 | agaggaaaaa aacttttgag acttttccgt tgccgctggg |
| | agccggaggc gcggggacct |
| 301 | cttggcgcga cgctgccccg cgaggaggca ggacttgggg |
| | accccagacc gcctcccttt |
| 361 | gccgccgggg acgcttgctc cctccctgcc ccctacacgg |
| | cgtccctcag gcgcccccat |
| 421 | tccggaccag ccctcgggag tcgccgaccc ggcctcccgc |
| | aaagactttt ccccagacct |
| 481 | cgggcgcacc ccctgcacgc cgccttcatc cccggcctgt |
| | ctcctgagcc cccgcgcatc |
| 541 | ctagaccctt tctcctccag gagacggatc tctctccgac |
| | ctgccacaga tcccctattc |
| 601 | aagaccaccc accttctggt accagatcgc gcccatctag |
| | gttatttccg tgggatactg |
| 661 | agacaccccc ggtccaagcc tcccctccac cactgcgccc |
| | ttctccctga ggagcctcag |
| 721 | ctttccctcg aggccctcct accttttgcc gggagacccc |
| | cagcccctgc aggggcgggg |
| 781 | cctccccacc acaccagccc tgttcgcgct ctcggcagtg |
| | ccggggggcg ccgcctcccc |
| 841 | catgccgccc tccgggctgc ggctgctgcc gctgctgcta |
| | ccgctgctgt ggctactggt |
| 901 | gctgacgcct ggcccgccgg ccgcgggact atccacctgc |
| | aagactatcg acatggagct |
| 961 | ggtgaagcgg aagcgcatcg aggccatccg cggccagatc |
| | ctgtccaagc tgcggctcgc |
| 1021 | cagccccccg agccaggggg aggtgccgcc cggcccgctg |
| | cccgaggccg tgctcgccct |
| 1081 | gtacaacagc acccgcgacc gggtggccgg ggagagtgca |
| | gaaccggagc ccgagcctga |
| 1141 | ggccgactac tacgccaagg aggtcacccg cgtgctaatg |
| | gtggaaaccc acaacgaaat |
| 1201 | ctatgacaag ttcaagcaga gtacacacag catatatatg |
| | ttcttcaaca catcagagct |
| 1261 | ccgagaagcg gtacctgaac ccgtgttgct ctcccgggca |
| | gagctgcgtc tgctgaggag |
| 1321 | gctcaagtta aaagtggagc agcacgtgga gctgtaccag |
| | aaatacagca acaattcctg |
| 1381 | gcgatacctc agcaaccggc tgctggcacc cagcgactcg |
| | ccagagtggt tatcttttga |
| 1441 | tgtcaccgga gttgtgcggc agtggttgag ccgtggaggg |
| | gaaattgagg gctttcgcct |
| 1501 | tagcgcccac tgctcctgtg acagcaggga taacacactg |
| | caagtggaca tcaacgggtt |
| 1561 | cactaccggc cgccgaggtg acctggccac cattcatggc |
| | atgaaccggc ctttcctgct |
| 1621 | tctcatggcc accccgctgg agagggccca gcatctgcaa |
| | agctcccggc accgccgagc |
| 1681 | cctggacacc aactattgct tcagctccac ggagaagaac |
| | tgctgcgtgc ggcagctgta |
| 1741 | cattgacttc cgcaaggacc tcggctggaa gtggatccac |
| | gagcccaagg gctaccatgc |
| 1801 | caacttctgc ctcgggccct gcccctacat ttggagcctg |
| | gacacgcagt acagcaaggt |
| 1861 | cctggccctg tacaaccagc ataacccggg cgcctcggcg |
| | gcgccgtgct gcgtgccgca |
| 1921 | ggcgctggag ccgctgccca tcgtgtacta cgtgggccgc |
| | aagcccaagg tggagcagct |
| 1981 | gtccaacatg atcgtgcgct cctgcaagtg cagctgaggt |
| | cccgccccgc cccgccccgc |
| 2041 | cccggcaggc ccggccccac cccgccccgc ccccgctgcc |
| | ttgcccatgg gggctgtatt |
| 2101 | taaggacacc gtgccccaag cccacctggg gccccattaa |
| | agatggagag aggactgcgg |
| 2161 | atctctgtgt cattgggcgc ctgcctgggg tctccatccc |
| | tgacgttccc ccactcccac |
| 2221 | tccctctctc tccctctctg cctcctcctg cctgtctgca |
| | ctattccttt gcccggcatc |
| 2281 | aaggcacagg ggaccagtgg ggaacactac tgtagttaga |
| | tctatttatt gagcaccttg |
| 2341 | ggcactgttg aagtgcctta cattaatgaa ctcattcagt |
| | caccatagca acactctgag |
| 2401 | atggcaggga ctctgataac acccatttta aaggttgagg |
| | aaacaagccc agagaggtta |
| 2461 | agggaggagt tcctgcccac caggaacctg ctttagtggg |
| | ggatagtgaa gaagacaata |
| 2521 | aaagatagta gttcaggcca ggcggggtgc tcacgcctgt |
| | aatcctagca cttttgggag |
| 2581 | gcagagatgg gaggatactt gaatccaggc atttgagacc |
| | agcctgggta acatagtgag |
| 2641 | accctatctc tacaaaacac ttttaaaaaa tgtacacctg |
| | tggtcccagc tactctggag |
| 2701 | gctaaggtgg gaggatcact tgatcctggg aggtcaaggc |
| | tgcag |
| // | |

Bottom of Form

Revised: Oct. 24, 2001.

Blast search in NCBI database using some tags from Example 6. Only the hit with the highest score is shown:

Tag Sequence for Query:

GTGGTGTGCGTGTCGAAGGT

Result:

Score E
   Sequences Producing Significant Alignments: (Bits) Value

gi|265568|gb|S54914.1| Mus musculus BUP (bup) gene, complete . . . 40 0.0071

[symbol]

gi|443026|emb|AL928680.5| Mouse DNA sequence from clone R . . . 40 0.007

gi|22797896|emb|AL158211.29| Human DNA sequence from clone . . . 40 0.007

[symbol] >gi|265568|gb|S54914.1| Mus musculus BUP (bup) gene, complete cds
Length=2022

Score=40.1 bits (20), Expect 0.007

Identities=20/20 (100%)

Strand=Plus/Plus

| | |
|---|---|
| Query: 1 | gtggtgtgcgtgtcgaaggt 20 |
| | \|\|\|\|\|\|\|\|\|\|\|\|\|\|\|\|\|\|\|\| |
| Sbjct: 968 | gtggtgtgcgtgtcgaaggt 987 |

[symbol] >gi|24430261|emb|AL928680.5| [symbol] Mouse DNA sequence from clone RP23-396N6 on chromosome 2, complete sequence
Length=217726

Score=40.1 bits (20), Expect=0.007

Identities=20/20 (100%)

Strand=Plus/Plus

| | |
|---|---|
| Query: 1 | gtggtgtgcgtgtcgaaggt 20 |
| | \|\|\|\|\|\|\|\|\|\|\|\|\|\|\|\|\|\|\|\| |
| Sbjct: 19552 | gtggtgtgcgtgtcgaaggt 19571 |

[symbol] >gi|22797896|emb|AL158211.29| [symbol] Human DNA sequence from clone RP11-573G6 on chromosome 10, complete sequence
Length=138094

Score=40.1 bits (20), Expect=0.007

Identities=20120 (100%)

Strand=Plus/Plus

| | |
|---|---|
| Query: 1 | gtggtgtgcgtgtcgaaggt 20 |
| | \|\|\|\|\|\|\|\|\|\|\|\|\|\|\|\|\|\|\|\| |
| Sbjct: 71390 | gtggtgtgcgtgtcgaaggt 71409 |

Tag Sequence for Query:

GACGCGGAAGGCGCGGCGGC

Result:

Score E
   Sequences Producing Significant Alignments: (Bits) Value

gi|8913518|gb|BC048682.1| Mus musculus, dystrobrevin bindi . . . 40 0.007

[symbol]

>gi|28913518|gb|BC048682.1| [symbol] Mus musculus, dystrobrevin binding protein 1, clone IMAGE:6515997, mRNA, partial cds
Length=1384

Score=40.1 bits (20), Expect=0.007

Identities=20/20 (100%)

Strand=Plus/Plus

| | | |
|---|---|---|
| Query: 1 | gacgcggaaggcgcggcggc | 20 |
| | \|\|\|\|\|\|\|\|\|\|\|\|\|\|\|\|\|\|\|\| | |
| Sbjct: 36 | gacgcggaaggcgcggcggc | 55 |

### Example 7

### Mapping of 5′ End Specific Tags to the Genome

5′ end specific sequence tags obtained as describe in this Example can be used to identify transcribed regions within genomes for which partial or entire sequences were obtained. Such a search can be performed using standard software solutions like NCBI BLAST (http://www.ncbi.nlm.nih.gov/BLAST/) to align the 5′ end specific sequence tags to genomic sequences. In the case of large genomes like those from human, rat or mouse it may be necessary to extend the initial sequence information obtained from concatemers. The use of extended sequences allows for a more precise identification of actively transcribed regions in the genome.

In another example 5′ end tags from concatemers prepared according to Examples 1 and 3 were further analyzed by mapping to the mouse genome. For this example a library of 5′ end tags was prepared from total brain of adult mice according to Example 1 and from 17.5 days whole embryos from mouse according to Example 3. Tag sequences were obtained from sequence reads by computational means as described in Example 5. Sequence tags were mapped to the mouse genome with a threshold of at least 18 bp matches and using penalties for mismatches or gaps. The table given below summarizes the results:

| Type | # Tags Used | Mapped | Single Site | Redundancy |
|---|---|---|---|---|
| Example 1 | 8,624 | 5,185 | 4,308 | 3,401 |
| Example 3 | 3,005 | 2,313 | 1,836 | 283 |

Statistical analysis and comparison to know genes indicated that about 89% of the sites are most likely true start sites of transcription.

### Example 8

### Statistical Analysis of 5′ End Sequence Tags

5′ end sequence tags obtained from the same plurality of mRNAs in a sample or nucleic acid fragments within the same cDNA library can be analyzed by a standard software solution like. NCBI BLAST (http://www.ncbi.nlm.nih.gov/BLAST/) to identify non-redundant sequence tags as describe in Example 5. All such non-redundant sequence tags can then be individually counted and further analyzed for the contribution of each non-redundant tag to the total number of all tags obtained from the same sample. The contribution of an individual tag to the total number of all tags should allow for a quantification of the transcripts in a plurality of mRNAs in the sample or a cDNA library. The results obtained in such a way on individual samples can be further compared with similar data obtained from other samples to compare their expression patterns.

### Example 9

### Identification of Transcriptional Start Sites

5′ end specific sequence tags, which could be mapped to genomic sequences, allow for the identification of regulatory sequences. In a gene the DNA upstream of the 5′ end of transcribed regions usually encompasses most of the regulatory elements, which are used in the control of gene expression. These regulatory sequences can be further analyzed for their functionality by searches in databases, which hold information on binding sites for transcription factors. Publicly available databases on transcription factor binding sites and for promoter analysis include:

Transcription Regulatory Region Database (TRRD) (http://wwwmgs.bionet.nsc.ru/mgs/dbases/trrd4/)

TRANSFAC (http://transfac.gbf.de/TRANSFAC/)

TFSEARCH (http://www.cbrc.jp/research/db/TFSEARCH.html)

PromoterInspector provide by Genomatix Software (http://www.genomatix.de/)

### Example 10

### Cloning of Full-Length cDNAs Using Information Derived from 5′ End Sequence Tags

Sequence information derived from the concatemers can be used to synthesize specific primers for the cloning of full-length cDNAs. In such an approach, the sequence derived from a given 5′ end, specific tag can be used to design a forward primer while the choice of the reverse primer would be dependent on the template DNA used in the amplification reaction. Amplification by the polymerase chain reaction (PCR) can be performed using a template derived from a plurality of RNA obtained from a biological sample and an oligo-dT primer. In the first step the oligo-dT primer and a reverse transcriptase are used to synthesize a cDNA pool. In the second step a forward primer derived from a 5′ end specific tag and an oligo-dT primer are used to amplify a full-length cDNA from the cDNA pool. Similarly, a specific full-length cDNA can be amplified from an exiting cDNA library using a forward primer derived from a 5′ end tag and a vector nested reversed primer.

### Example 11

### Alternative Approaches for the Cloning of 5′-End Tags from cDNA Libraries

A plurality of cDNAs can be amplified from an exciting cDNA library having a recognition site for a class IIs endonuclease at the 5′ end of the inserts. The PCR products derived from such a library would be further treated as described in the examples herein.

### Example 12

### Cloning of 5′ Ends by Replacement of the Cap Structure by an Oligonucleotide Having a Class IIs Recognition Site

A cDNA/RNA hybrid encompassing the 5′ end of an initial transcript can be obtained as described in Examples 1 to 3. The Cap structure in such cDNA/RNA hybrids is then enzymatically removed by a hydrolyzing enzyme such as the T4 polynucleotide kinase or the tobacco acid pyrophosphatase. A single or double-stranded oligonucleotide having a class IIs recognition site is then ligated by T4 RNA ligase to the RNA at the phosphate present at the 5′ end of the de-capped mRNA. The ligated oligonucleotide will function as a primer for the second strand synthesis following the procedure given in Examples 1 to 3. By the use of a modified oligonucleotide in the ligation step the double-stranded cDNA can be attached to a support and used for the cloning of concatemers as described herein.

### Example 13

### Amplification Step for a Sample

In cases where the amount of a sample is limiting to the invention, the sample material can be amplified by the following approach. In a first step a plurality of mRNAs is treated as described in Example 11 to replace the cap structure by an appropriate oligonucleotide having a class IIs recognition site. In a second step the aforementioned template is amplified by a PCR step using a primer complementary to the linker and a poly-A primer. The PCR product can be used for the invention as described in the Examples 1.

### Example 14

### Utilization of Extended 5′-End Sequences

Initial 5′ end sequences obtained for concatemers can be used to synthesize sequencing primers to obtain extended sequence information on the 5′ end of a transcribed region.

### Example 15

### Gene Inactivation

Sequence information obtained from 5′ end specific sequence tags can be used for the design of anti-sense probes or RNAi, which could be applied in knockdown studies.

### REFERENCES

Velculescu V E, Zhang L, Vogelstein B, Kinzler K W, Serial analysis of gene expression, Science 1995 Oct. 20; 270(5235):484-7
U.S. Pat. No. 5,866,330 (SAGE)
U.S. Pat. No. 5,695,937 (SAGE)
U.S. patent publication No. 20030008290 (LongSAGE)
U.S. patent publication No. 20030049653 (LongSAGE)
Piero Carninci et al., Methods in Enzymology, Vol. 303, pp. 1944, 1999
U.S. Pat. No. 6,013,488 (RIKEN)
Lee S, Clark T, Chen J, Zhou G, Scott L R, Rowley J D, Wang S M, Correct identification of genes from serial analysis of gene expression tag sequences, Genomics 2002 April; 79(4):598-602
Saha S, Sparks A B, Rago C, Akrnaev V, Wang C J, Vogelstein B, Kinzler K W, Velculescu V E, Using the transcriptome to annotate the genome, Nat Biotechnol 2002 May; 20(5):508-12
Maruyama K and Sugano S, Oligo-capping: a simple method to replace the cap structure of eukaryotic mRNAs with oligoribonucleotides. Gene. 1994, Vol. 138:171-4
Suzuki Y, Taira H, Tsunoda T, Mizushima-Sugano J, Sese J, Hata H, Ota T, Isogai T, Tanaka T, Morishita S, Okubo K, Sakaki Y, Nakamura Y, Suyama A, Sugano S. Links Diverse transcriptional initiation revealed by fine, large-scale mapping of mRNA start sites. EMBO Rep. 2001 May; 2(5):388-93.
Suzuki Y, Tsunoda T, Sese J, Taira H, Mizushima-Sugano J, Hata H, Ota T, Isogai T, Tanaka T, Nakamura Y, Suyama A, Sakaki Y, Morishita S, Okubo K, Sugano S. Links Identification and characterization of the potential promoter regions of 1031 kinds of human genes. Genome Res. 2001 May; 11(5):677-84.
Theissen H, Etzerodt M, Reuter R, Schneider C, Lottspeich F, Argos P, Luhrann R, Philipson L. Cloning of the human cDNA for the U1 RNA-associated 70K protein. EMBO J. 1986 Dec. 1; 5(12):3209-17
Edery I, Chu L L, Sonenberg N, Pelletier J, An efficient strategy to isolate full-length cDNAs based on an mRNA cap retention procedure (CAPture), Mol Cell Biol 1995 June; 15(6):3363-71
U.S. Pat. No. 6,022,715 (GenSet)
Carninci P, Nakamura M, Sato K, Hayashizaki Y, Brownstein M J., Cytoplasmic RNA extraction from fresh and frozen mammalian tissues, Biotechniques 2002 August; 33(2):306-9
Shibata Y, Carninci P, Watahiki A, Shiraki T, Konno H, Muramatsu M, Hayashizaki Y, Cloning full-length, cap-trapper-selected cDNAs by using the single-strand linker ligation method, Biotechniques 2001 June; 30(6):1250-4
Sambrook J and Russel D W, Molecular Cloning A Laboratory Manual, Cold Spring Harbor Laboratory Press, New York, 2001
Carninci P, Shibata Y, Hayatsu N, Itoh M, Shiraki T, Hirozane T, Watahiki A, Shibata K, Konno H, Muramatsu M, Hayashizaki Y, Balanced-size and long-size cloning of full-length, cap-trapped cDNAs into vectors of the novel lambda-FLC family allows enhanced gene discovery rate and functional analysis, Genomics. 2001 September; 77(1-2):79-90.
Heinemeyer T, Wingender E, Reuter I, Hermjakob H, Kel A E, Kel O V, Ignatieva E V, Ananko E A, Podkolodnaya O A, Kolpakov F A, Podkolodny N L, Kolchanov N A, Databases on transcriptional regulation: TRANSFAC, TRRD and COMPEL, Nucleic Acids Res 1998 Jan. 1; 26(1):362-7
Maruyama K, Sugano S. Oligo-capping: a simple method to replace the cap structure of eukaryotic mRNAs with oligoribonucleotides. Gene. 1994 Jan. 28; 138(1-2):171-4.
Jordan B., DNA Microarrays: Gene Expression Applications, Springer-Verlag, Berlin Heidelberg New York, 2001
Schena A, DNA Microarrays, A Practical Approach, Oxford University Press, Oxford 1999
U.S. Pat. No. 5,962,272 (Clontech)
Carninci P, Shiraki T, Mizuno Y, Muramatsu M, Hayashizaki Y, Extra-long first-strand cDNA synthesis, Biotechniques 2002 May; 32(5): 984-5
U.S. Pat. Nos. 6,352,828; 6,306,597; 6,280,935; 6,265,163; and 5,695,934 (Lynx)
Itoh M. et al. 1997, Nucleic Acids Res 25:1315-1316
Itoh M. et al. 1999, Genome Res. 9-463-470
Shibata K. et al. 2000, Genome Res. 10, 1757-71

1. A method for preparing a DNA fragment corresponding to a nucleotide sequence of a 5′ end region of an mRNA, comprising the steps of:
(a) preparing a nucleic acid corresponding to a nucleotide sequence of the 5′ end of an mRNA;
(b) attaching at least one linker to the nucleic acid;
(c) cleaving the nucleic acid with a restriction enzyme having its recognition site within the linker and its cleavage site within the nucleic acid corresponding to the 5′ end of the mRNA; and
(d) collecting a resulting DNA fragment corresponding to the 5′ end of the mRNA.

2. The method according to claim 1, wherein the length of the DNA fragment is about 5-100 bp.

3. The method according to claim 1, wherein the length of the DNA fragment is about 15-30 bp.

4. The method according to claim 1, wherein the length of the DNA fragment are about 10-30 bp.

5. The method according to claim 1, wherein the nucleic acid in step (a) is derived from one selected from the group consisting of a total RNA, an mRNA and a full-length cDNA.

6. The method according to claim 1, wherein step (a) comprises the steps of:
substituting a 5′ cap structure of the mRNA with an oligonucleotide; and
synthesizing a first-strand cDNA using the mRNA as a template to produce a nucleic acid corresponding to the 5′ end of the mRNA.

7. A method for preparing a DNA fragment corresponding to a nucleotide sequence of a 5′ end region of an mRNA, comprising steps of:
(a) substituting a cap structure of an mRNA with an oligonucleotide, wherein the oligonucleotide comprises a restriction enzyme recognition site, and a cleavage site of a restriction enzyme is within the nucleic acid corresponding to the 5′ end of the mRNA;
(b) synthesizing a first strand cDNA using the mRNA as a template;
(c) synthesizing a second strand cDNA using the first stand cDNA as a template;
(d) cleaving a resulting double stranded cDNA with the restriction enzyme; and
(e) collecting a resulting DNA fragment corresponding to 5′ end of the mRNA.

8. The method according to claim 1 or 7, wherein the nucleic acid in step (a) is derived from a biological sample, an in vitro synthesized RNA, a cDNA library, artificially created pluralities of nucleic acids, or a tag library.

9. The method according to claim 1, wherein step (a) comprises the steps of:
synthesizing first-strand cDNAs using RNAs as a template and producing cDNA/RNA hybrids of the resulting first-strand cDNAs and the RNAs;
selecting a particular cDNA/RNA hybrid that has the 5′ cap structure of the mRNA using a selective binding substance which specifically recognizes the 5′ cap structure; and
recovering a nucleic acid corresponding to the 5′ end of the mRNA.

10. The method according to claim 9, wherein the nucleic acid prepared in step (a) is a full-length cDNA, wherein the selective binding substance is attached to a support.

11. The method according to claim 1, wherein step (a) comprises the steps of:
synthesizing first strand cDNAs using RNAs as a template and producing cDNA/RNA hybrids of the resulting first strand cDNAs and the RNAs; and
recovering a nucleic acid corresponding to the 5′ end region of the mRNA from the cDNA/RNA hybrids.

12. The method according to claim 1, wherein step (a) comprises the steps of:
synthesizing first strand cDNAs using RNAs as a template and producing cDNA/RNA hybrids of the resulting first strand cDNAs and the RNAs;
conjugating a selective binding substance to a 5′ cap structure of an mRNA present in the RNAs;
contacting the cDNA/RNA hybrids with a support, wherein another matching selective binding substance is fixed to the support, and the matching selective binding substance specifically binds to the selective binding substance; and
recovering the a nucleic acid corresponding to the 5′ end of the mRNA from the mRNA fixed to the support.

13. The method according to claim 9 or 10, wherein the selective binding substance is a cap binding protein or a cap binding antibody.

14. The method according to claim 12, wherein the selective binding substance is biotin, and the matching selective binding substance is selected from the group consisting of avidin, streptavidin and a derivative therefrom which specifically binds to biotin.

15. The method according to claim 12, wherein the selective binding substance is digoxigenin and the matching selective binding substance is an antibody against digoxigenin.

16. The method according to claim 10 or 12, wherein the support is made of magnetic beads, agarose beads, latex beads, sepharose matrix, silicagel matrix or glass beads.

17. The method according to claim 1, wherein step (b) comprises the steps of:
attaching a linker to an end region corresponding to the nucleotide sequence of a 5′ end region of the mRNA, wherein the linker carries at least one restriction enzyme recognition site for a restriction enzyme that cleaves a site different from its recognition sequence;
synthesizing a second-strand cDNA using the nucleic acid as a template;
treating a resulting linker-bound double-stranded cDNA with the restriction enzyme; and
recovering a resulting fragment which contains a linker moiety and a part of cDNA corresponding to the 5′ end regions of the mRNA.

18. The method according to claim 17, wherein the linker contains a double-stranded oligonucleotide region, and the second-strand cDNA is synthesized using the linker.

19. The method according to claim 17, wherein the second-strand cDNA is synthesized using other oligonucleotides which are partially or totally complement to the linker.

20. The method according to claim 17, wherein a selective binding substance is attached to or included in the linker, and the recovering step comprises the steps of binding the selective binding substance to a matching selective binding substance immobilized on a support, and recovering the support, wherein the matching selective binding substance specifically binds to the selective binding substance.

21. The method according to claim 20, wherein the selective binding substance is biotin, and the matching selective binding substance is selected from the group consisting of avidin, streptavidin and a derivative therefrom which specifically binds to biotin.

22. The method according to claim 20, wherein the selective binding substance is digoxigenin, and the matching selective binding substance is an antibody against digoxigenin.

23. The method according to claim 17, wherein the restriction enzyme is the Class II or Class III restriction enzyme.

24. The method according to claim 17, wherein the restriction enzyme is the Class IIG and Class IIS restriction enzymes.

25. The method according to claim 23, wherein the restriction enzyme is selected from the group consisting of Gsu I, MmeI, BpmI, BsgI and EcoP15I.

26. A method for determining a nucleotide sequence of the 5′ end region of the mRNA by sequencing the DNA fragment prepared by the method according to claim 1.

27. The method according to claim 1, further comprising amplifying the nucleic acid corresponding the 5′ end region of the mRNA by a DNA polymerase or a cocktail of DNA polymerases.

28. The method according to claim 27, wherein the DNA polymerase is heat-stable.

29. The method according to claim 27, wherein the DNA polymerase is selected from the group consisting of Taq polymerase, Pwo DNA polymerase, Kod DNA polymerase, Pfu DNA polymerase, Vent DNA polymerase, Deep Vent DNA polymerase, rBST DNA polymerase, and Master Amp AmpliTherm DNA polymerase.

30. The method according to claim 1, wherein the first strand cDNA is synthesized and fractionated by physical means.

31. The method according to claim 30, wherein the nucleic acid is fractionated by hybridizing to a plurality of nucleic acids.

32. A method according to claim 1, further comprising the step of attaching the collected nucleic acid to beads.

33. A method for preparing a concatemer comprising one or more DNA fragments, comprising the step of ligating one or more of DNA fragments obtained by the method according to claim 1 and corresponding to the 5′ end of the mRNA.

34. A concatemer prepared by the method according to claim 33.

35. A vector comprising the concatemer according to claim 34.

36. A sequence derived from the concatemer according to claim 34.

37. The method for determining the transcriptional states of a sample using a sequence derived from the DNA fragment prepared by the method according to claim 1.

38. The method for obtaining expression data on a plurality of mRNAs or cDNAs in a sample using a sequence derived from the DNA fragment prepared by the method according to claim 1.

39. The method quantifying expression data on a plurality of mRNAs in a sample using a sequence derived from the DNA fragment prepared by the method according to claim 1.

40. The method for building a database holding sequence information using a sequence derived from the DNA fragment prepared by the method according to claim 1.

41. The method identifying transcribed regions from a genomic sequence using a sequence derived from the DNA fragment prepared by the method according to claim 1.

42. The method for identifying a transcription initiation site and a related regulatory sequence in a genomic sequence using a sequence derived from the DNA fragment prepared by the method according to claim 1.

43. The method for cloning a full-length or partial cDNA from a cDNA library or biological sample using a sequence derived from the DNA fragment prepared by the method according to claim 1.

44. The method for cloning a complete or partial promoter region of a gene from a genomic library or genomic DNA using a sequence derived from the DNA fragment prepared by the method according to claim 1.

45. The method for analyzing the activity of regulatory regions in a genome based on genomic sequence information using a sequence derived from the DNA fragment prepared by the method according to claim 1.

46. The method for inactivating a gene or altering its expression using a sequence derived from the DNA fragment prepared by the method according to claim 1.

47. The method according to claim 46, wherein the gene is inactivated or altered in its expression by the means of siRNA or RNAi.

48. The method for synthesizing a nucleotide sequence to be used as the linker or primer based on a sequence derived from the DNA fragment prepared by the method according to claim 1.

49. The method for synthesizing a hybridization probe based on a sequence derived from the DNA fragment prepared by the method according to claim 1.

50. The method according to claim 49, wherein the hybridization probe is attached to a support.

51. The method according to claim 49, wherein the hybridization probe is a probe to identify the sequence corresponding to the nucleotide sequence of the 5′ end region of the mRNA.

52. The method according to claim 1, further comprising extending the 5′ end region of the nucleotide sequence.

53. A method according to claim 1 used for the development of diagnostic tools.

54. A method according to claim 1 used for the development of research tools.

55. A method according to claim 1 used for the development of a reagent or a kit.

56. The method according to claim 7, wherein the nucleic acid in step (a) is derived from a biological sample, an in vitro synthesized RNA, a cDNA library, artificially created pluralities of nucleic acids, or a tag library.

57. The method according to claim 10, wherein the selective binding substance is a cap binding protein or a cap binding antibody.

58. The method according to claim 12, wherein the support is made of magnetic beads, agarose beads, latex beads, sepharose matrix, silicagel matrix or glass beads.


SEQUENCE LISTING (77 sequences)

SEQ ID NO: 1 - DNA, 74 bp, Artificial
  First strand cDNA primer
gagagagagaaaggatcctgccatttcattacctctttctccgcacccgacatagattttttttttttttttvn

SEQ ID NO: 2 - DNA, 60 bp, Artificial
  Upper oligonucleotide GN5
agagagagacctcgagtaactataacggtcctaaggtagcgacctaggtccgacgnnnnn

SEQ ID NO: 3 - DNA, 60 bp, Artificial
  Upper oligonucleotide N6
agagagagacctcgagtaactataacggtcctaaggtagcgacctaggtccgacnnnnnn

SEQ ID NO: 4 - DNA, 54 bp, Artificial
  Lower oligonucleotide
gtcggacctaggtcgctaccttaggaccgttatagttactcgaggtctctctct

SEQ ID NO: 5 - DNA, 55 bp, Artificial
  primer
agagagagacctcgagtaactataacggtcctaaggtagcgacctaggtccgacg

SEQ ID NO: 6 - DNA, 45 bp, Artificial
  linker
tctagatcaggactcttctatagtgtcacctaaagtctctctctc

SEQ ID NO: 7 - DNA, 47 bp, Artificial
  linker
gagagagagactttaggtgacactatagaagagtcctgatctagann

SEQ ID NO: 8 - DNA, 25 bp, Artificial
  Primer 1 (uni-PCR)
gagagagagactttaggtgacacta

SEQ ID NO: 9 - DNA, 25 bp, Artificial
  Primer 2(MmeI-PCR)
agagagagacctcgagtaactataa

SEQ ID NO: 10 - DNA, 44 bp, Artificial
  first strand oligo-dT primer
gagagagagaggatccttctggagagttttttttttttttttvn

SEQ ID NO: 11 - DNA, 45 bp, Artificial
  Oligonucleotide Bg-Gsu-GN5
agagagagaactaggcttaataggtgactagatctggaggnnnnn

SEQ ID NO: 12 - DNA, 45 bp, Artificial
  Oligonucleotide Bg-Gsu-N6
agagagagaactaggcttaataggtgactagatctggagnnnnnn

SEQ ID NO: 13 - DNA, 39 bp, Artificial
  Oligonucleotide Bg-Gsu-down
ctggagatctagtcacctattaagcctagttctctctct

SEQ ID NO: 14 - DNA, 47 bp, Artificial
  Oligonucleotide Bg-Mme-GN5
agagagagaactaggcttaataggtgactagatcttccracgnnnnn

SEQ ID NO: 15 - DNA, 47 bp, Artificial
  Oligonucleotide Bg-Mme-N6
agagagagaactaggcttaataggtgactagatcttccracnnnnnn

SEQ ID NO: 16 - DNA, 40 bp, Artificial
  Oligonucleotide Bg-Mme-down
gtyggagatctagtcacctattaagcctagttctctctct

SEQ ID NO: 17 - DNA, 47 bp, Artificial
  oligonucleotide
gagagagagactttaggtgacactatagaagagtcctgagaattcnn

SEQ ID NO: 18 - DNA, 45 bp, Artificial
  oligonucleotide
gaattctcaggactcttctatagtgtcacctaaagtctctctctc

SEQ ID NO: 19 - DNA, 49 bp, Artificial
  linker
agagagagagcttagatgagagtgactcgagcctaggtccaacgnnnnn

SEQ ID NO: 20 - DNA, 49 bp, Artificial
  linker
agagagagagcttagatgagagtgactcgagcctaggtccaacnnnnnn

SEQ ID NO: 21 - DNA, 43 bp, Artificial
  linker
gttggacctaggctcgagtcactctcatctaagctctctctct

SEQ ID NO: 22 - DNA, 42 bp, Artificial
  second linker
gaattctacgcctctcgatcgaaatcccgatctaggctagcg

SEQ ID NO: 23 - DNA, 42 bp, Artificial
  second linker
cttaagatgcggagagcgtgaatcgagtttaaggctagcatc

SEQ ID NO: 24 - DNA, 25 bp, Artificial
  primer
ttagatgagagtgactcgagcctag

SEQ ID NO: 25 - DNA, 25 bp, Artificial
  primer
ctacgatcggaatttgagctaagtg

SEQ ID NO: 26 - DNA, 17 bp, Artificial
  primer
caggaaacagctatgac

SEQ ID NO: 27 - DNA, 16 bp, Artificial
  primer
gtaaaacgacggccag

SEQ ID NO: 28 - DNA, 596 bp, Homo sapiens
  misc_feature (432)..(432): “n” is any nucleotide
tcgttaactattaggcgaattgggccctctaggtcgacgagttctcagcagagccgccgtctagagccccgccctcccgggccaccgtcggacctagaatagttactcgaggtctctcgtcggacctagagtttttcgtatgtttgtcatcgtcggacctaggtccgacggtccattcctgagagtctctctaggtccgacgagagagagaggatccttctgtctagaccctgacgccggaaccgcaccgtcggacctaggtccgacggaaaagcagcttcctccactctaggtccgacggtgtgtgtgtgtgtgcgtgttctagagactggttcagatcaaaagtcgtcggacctaggtccgacggggctggtgagatggctcagtctagatgcatgctcgagcggccgccagtgtgatggatatctgccnaatnccagcacaccggcgcgcgcnaccagtggatccgagcccggtaccaagcttgatgcatacctcgagtatcctatactgtcacctaaatagcttggggtaatcatggtcatagctgtctcctgtgtgaaattgttatccgctcaaaattcccaacaacatag

SEQ ID NO: 29 - DNA, 12 bp, Artificial
  linker
nctaggtccgac

SEQ ID NO: 30 - DNA, 20 bp, Artificial
  linker
ngttggacctaggtccaacn

SEQ ID NO: 31 - DNA, 13 bp, Artificial
  linker
tctaggtccgacg

SEQ ID NO: 32 - DNA, 13 bp, Artificial
  linker
cctaggtccgacg

SEQ ID NO: 33 - DNA, 20 bp, Artificial
  tag1
gtggcccgggagggcggggc

SEQ ID NO: 34 - DNA, 19 bp, Artificial
  tag2
agagacctcgagtaactat

SEQ ID NO: 35 - DNA, 20 bp, Artificial
  tag3
atgacaaacatacgaaaaac

SEQ ID NO: 36 - DNA, 19 bp, Artificial
  tag4
gtccattcctgagagtctc

SEQ ID NO: 37 - DNA, 20 bp, Artificial
  tag5
agagagagaggatccttctg

SEQ ID NO: 38 - DNA, 20 bp, Artificial
  tag6
gtgcggttccggcgtcaggg

SEQ ID NO: 39 - DNA, 19 bp, Artificial
  tag7
gaaaagcagcttcctccac

SEQ ID NO: 40 - DNA, 20 bp, Artificial
  tag8
gtgtgtgtgtgtgtgcgtgt

SEQ ID NO: 41 - DNA, 20 bp, Artificial
  tag9
acttttgatctgaaccagtc

SEQ ID NO: 42 - DNA, 20 bp, Artificial
  tag10
gggctggtgagatggctcag

SEQ ID NO: 43 - DNA, 19 bp, Artificial
  tag1
gtacctcctcgcatcccgc

SEQ ID NO: 44 - DNA, 20 bp, Artificial
  tag2
gtggtgtgcgtgtcgaaggt

SEQ ID NO: 45 - DNA, 20 bp, Artificial
  tag3
atggaccgagggccccagcc

SEQ ID NO: 46 - DNA, 19 bp, Artificial
  tag4
cggatcgggtgggtcggac

SEQ ID NO: 47 - DNA, 19 bp, Artificial
  tag5
agaggtcgcagcagttcgt

SEQ ID NO: 48 - DNA, 20 bp, Artificial
  tag6
tctccggagccggcgctgtg

SEQ ID NO: 49 - DNA, 19 bp, Artificial
  tag7
agactttgcaggctccgag

SEQ ID NO: 50 - DNA, 20 bp, Artificial
  tag8
ggagctgccgcagcgccgga

SEQ ID NO: 51 - DNA, 20 bp, Artificial
  tag9
acaccgtcggacctggtcgc

SEQ ID NO: 52 - DNA, 20 bp, Artificial
  tag10
agacgttctcgcccagagtc

SEQ ID NO: 53 - DNA, 20 bp, Artificial
  tag11
gccgttccttgcttgctgga

SEQ ID NO: 54 - DNA, 20 bp, Artificial
  tag12
gggttggggatttagctcag

SEQ ID NO: 55 - DNA, 19 bp, Artificial
  tag13
gagtaactataacggtcct

SEQ ID NO: 56 - DNA, 19 bp, Artificial
  tag14
gattccgcctggagctcgc

SEQ ID NO: 57 - DNA, 18 bp, Artificial
  tag15
agggaccgctgcggtccg

SEQ ID NO: 58 - DNA, 18 bp, Artificial
  zzb21106i09t3 junk
cattaggggattgggccc

SEQ ID NO: 59 - DNA, 28 bp, Artificial
  zzb21106i09t3 junk
acccggggggcgggactaaccgtcggac

SEQ ID NO: 60 - DNA, 20 bp, Artificial
  tag1
gacgcggaaggcgcggcggc

SEQ ID NO: 61 - DNA, 21 bp, Artificial
  tag2
ggagggcgggcggcggccctc

SEQ ID NO: 62 - DNA, 19 bp, Artificial
  tag3
caaaaaaaaaaaaaaaact

SEQ ID NO: 63 - DNA, 20 bp, Artificial
  tag4
aggctctgctcgctctgccc

SEQ ID NO: 64 - DNA, 19 bp, Artificial
  tag5
acttctgattctgacagac

SEQ ID NO: 65 - DNA, 19 bp, Artificial
  tag6
acagtggcgtctgcaaagc

SEQ ID NO: 66 - DNA, 20 bp, Artificial
  tag7
ggaaagtccaggtggacttt

SEQ ID NO: 67 - DNA, 19 bp, Artificial
  tag8
gccgccgaggccgcgcagg

SEQ ID NO: 68 - DNA, 19 bp, Artificial
  tag9
gttagtgtataacagagtt

SEQ ID NO: 69 - DNA, 20 bp, Artificial
  tag10
tcgcccgctgttcagtctct

SEQ ID NO: 70 - DNA, 19 bp, Artificial
  tag11
aggtggggcaagatggctg

SEQ ID NO: 71 - DNA, 20 bp, Artificial
  tag12
ggcatggccagaaggcaagc

SEQ ID NO: 72 - DNA, 20 bp, Artificial
  tag13
gacgcacgcatagagggggg

SEQ ID NO: 73 - DNA, 19 bp, Artificial
  tag14
nccatggaacagccacact

SEQ ID NO: 74 - DNA, 26 bp, Artificial
  junk1
tgataaggcaatggcctctaatgctg

SEQ ID NO: 75 - DNA, 16 bp, Artificial
  tag
acctccctccgcggag

SEQ ID NO: 76 - DNA, 2745 bp, Homo sapiens
  CDS (842)..(2017)
acctccctccgcggagcagccagacagcgagggccccggccgggggcaggggggacgccccgtccggggcaccccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggcccgccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacaccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatggcagggactctgataacacccattttaaaggttgaggaaacaagcccagagaggttaagggaggagttcctgcccaccaggaacctgctttagtgggggatagtgaagaagacaataaaagatagtagttcaggccaggcggggtgctcacgcctgtaatcctagcacttttgggaggcagagatgggaggatacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcag

SEQ ID NO: 77 - protein, 391 aa, Homo sapiens
MPPSGLRLLPLLLPLLWLLVLTPGPPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS